SEQUENCE LISTING (20 sequences)

SEQ ID NO: 1 - protein, 5 aa, synthetic construct
  REGION 1..5: note = Synthetic Sequence
SYAMS

SEQ ID NO: 2 - protein, 17 aa, synthetic construct
  REGION 1..17: note = Synthetic Sequence
TISSGGFATYYPDTVKG

SEQ ID NO: 3 - protein, 11 aa, synthetic construct
  REGION 1..11: note = Synthetic Sequence
HGGGSYGWFAY

SEQ ID NO: 4 - protein, 11 aa, synthetic construct
  REGION 1..11: note = Synthetic Sequence
ITSTDIDDDMN

SEQ ID NO: 5 - protein, 7 aa, synthetic construct
  REGION 1..7: note = Synthetic Sequence
EGNTLRP

SEQ ID NO: 6 - protein, 9 aa, synthetic construct
  REGION 1..9: note = Synthetic Sequence
LQSDNMPYT

SEQ ID NO: 7 - protein, 139 aa, synthetic construct
  REGION 1..139: note = Synthetic Sequence
MNFGLSLIFLVLVLKGVQCEVQLVESGGDLVKPGGSLKLSCAVSGFTFSSYAMSWVRQTPAKRLEWVATISSGGFATYYPDTVKGRFTISRDNAKNTLYLQMSSLRSEDTAMYYCARHGGGSYGWFAYWGQGTLVTVSE

SEQ ID NO: 8 - protein, 127 aa, synthetic construct
  REGION 1..127: note = Synthetic Sequence
MLSLAPLLSLLLLCVSDSRAETTVTQSPASLSVATGEKVTIRCITSTDIDDDMNWYQQKPGEPPKLLISEGNTLRPGVPSRFSSSGYGTDFVFTIENTLSEDFADYYCLQSDNMPYTFGGGTKLEIK

SEQ ID NO: 9 - protein, 6 aa, synthetic construct
  REGION 1..6: note = Synthetic Sequence
PIAYMS

SEQ ID NO: 10 - protein, 17 aa, synthetic construct
  REGION 1..17: note = Synthetic Sequence
DILPSIGRTIYGEKFED

SEQ ID NO: 11 - protein, 9 aa, synthetic construct
  REGION 1..9: note = Synthetic Sequence
QDTYYAMDY

SEQ ID NO: 12 - protein, 10 aa, synthetic construct
  REGION 1..10: note = Synthetic Sequence
SASSSVSYMH

SEQ ID NO: 13 - protein, 7 aa, synthetic construct
  REGION 1..7: note = Synthetic Sequence
STSNLAS

SEQ ID NO: 14 - protein, 9 aa, synthetic construct
  REGION 1..9: note = Synthetic Sequence
QQRSSYPYT

SEQ ID NO: 15 - protein, 138 aa, synthetic construct
  REGION 1..138: note = Synthetic Sequence
MDWIWIMLHLLAAATGLQSQVHLQQSGSELRSPGSSVKLSCKDFDSEVFPIAYMSWVRQKPGHGFEWIGDILPSIGRTIYGEKFEDKATLDADTVSNTAYLELNSLTSEDSAIYYCARQDTYYAMDYWGQGTSVTVSS

SEQ ID NO: 16 - protein, 128 aa, synthetic construct
  REGION 1..128: note = Synthetic Sequence
MHFQVQIFSFLLISASVIMSRGQIVLTQSPAIMSASPGEKVTITCSASSSVSYMHWVQQKPGTSPKLWIYSTSNLASGVPARFRGSGSGTSYSLTISRMEAEDAATYYCQQRSSYPYTFGGGTKLEIK

SEQ ID NO: 17 - DNA, 417 bp, synthetic construct
  misc_feature 1..417: note = Synthetic Sequence
  source 1..417: mol_type = other DNA
atgaacttcgggctcagcttgattttccttgtccttgttttaaaaggtgtccagtgtgaagtgcagctggtggagtctgggggagacttagtgaagcctggagggtccctgaaactctcctgtgcagtttctggattcactttcagtagctatgccatgtcttgggttcgccagactccggcgaagaggctggagtgggtcgcaaccattagtagtggtggttttgccacctactatccagacactgtgaagggccgattcaccatctccagagacaatgccaaaaacaccctgtacctgcaaatgagcagtctgaggtctgaggacacagccatgtattactgtgcaagacatgggggtggtagctacggctggtttgcttactggggccaagggactctggtcactgtctctgaa

SEQ ID NO: 18 - DNA, 381 bp, synthetic construct
  misc_feature 1..381: note = Synthetic Sequence
  source 1..381: mol_type = other DNA
atgctctcactagctcctctcctcagccttcttctcctctgtgtctctgattctagggcagaaacaactgtgacccagtctccagcatccctgtccgtggctacaggagaaaaagtcactatcagatgcataaccagcactgatattgatgatgatatgaactggtaccagcagaagccaggggaacctcctaagctccttatttcagaaggcaatactcttcgtcctggagtcccatcccgattctccagcagtggctatggcacagattttgtttttacaattgaaaacacgctctcagaagattttgcagattactactgtttgcaaagtgataacatgccgtacacgttcggaggggggaccaagctggaaataaaa

SEQ ID NO: 19 - DNA, 414 bp, synthetic construct
  misc_feature 1..414: note = Synthetic Sequence
  source 1..414: mol_type = other DNA
atggactggatttggatcatgctccatctgctggcagcagctacaggtctccaatcccaggttcacctacaacagtctggttctgaactgaggagtcctgggtcttcagtaaagctttcatgcaaggattttgattcagaagtcttccctattgcttatatgagttgggttaggcagaagcctggacatggatttgagtggattggtgacatactcccaagtattggtagaacaatctatggagagaagtttgaggacaaagccacactggatgcagacacagtgtccaacacagcctacttggagctcaacagtctgacatctgaggactctgctatctactactgtgcaagacaagacacttactatgctatggactactggggtcaaggaacctcagtcaccgtctcctca

SEQ ID NO: 20 - DNA, 384 bp, synthetic construct
  misc_feature 1..384: note = Synthetic Sequence
  source 1..384: mol_type = other DNA
atgcattttcaagtgcagattttcagcttcctgctaatcagtgcctcagtcataatgtccagaggacaaattgttctcacccagtctccagcaatcatgtctgcatctccaggggagaaggtcaccataacctgcagtgccagctcaagtgtaagttacatgcactgggtccagcagaagccaggcacttctcccaaactctggatttatagcacatccaacctggcttctggagtccctgctcgcttcagaggcagtggatctgggacctcttactctctcacaatcagccgaatggaggctgaagatgctgccacttattactgccagcaaaggagtagttacccgtacacgttcggaggggggaccaagctggaaataaaa

(19) United States
(12) Patent Application Publication
(10) Pub. No.: US 2024/0425583 A1
(43) Pub. Date: Dec. 26, 2024

(54) METHODS AND COMPOSITIONS RELATED TO MCL-1 AND BIM HETERODIMER ANTIBODIES

(71) Applicants: Michael H. CARDONE, Dorchester, MA (US); Andrew KINLOCH, Cambridge, MA (US)

(72) Inventors: Michael H. CARDONE, Dorchester, MA (US); Andrew KINLOCH, Cambridge, MA (US)

(21) Appl. No.: 18/292,658

(22) PCT Filed: Jul. 26, 2022
(86) PCT No.: PCT/US2022/074144

§ 371 Date: Jan. 26, 2024

Related U.S. Application Data
(60) Provisional application No. 63/225,697, filed on Jul. 26, 2021.

(51) Int. Cl. C07K 16/28; G01N 33/68

(52) U.S. Cl. CPC C07K 16/28; G01N 33/6854; C07K 2317/565; C07K 2317/567; G01N 2800/52

(57) ABSTRACT

The present disclosure relates to compositions and methods of determining cancer cell sensitivity to treatment using antibodies that detect heterodimers comprising Bcl-2 proteins selected from MCL-1 and BIM. The disclosure also provides methods for predicting a cancer patient's sensitivity to the cancer treatment.

### CROSS-REFERENCE TO RELATED APPLICATIONS

This application claims priority to U.S. Provisional Application No. 63/225,697, filed on Jul. 26, 2021, the entire contents of which are incorporated herein.

### DESCRIPTION OF THE TEXT FILE SUBMITTED ELECTRONICALLY

The content of the text file submitted electronically herewith is incorporated herein by reference in their entirety: A computer readable format copy of the Sequence Listing (Filename: “EUTR-023PC_105444-5023.xml”: Date recorded: Jul. 25, 2022: File size: 28,672 bytes in size).

### BACKGROUND

Treatment of cancer patients continues to be challenging due to the heterogeneity of tumors, and the absence of well-defined molecular targets. As more therapies are approved for different types of cancer, predictive and prognostic biomarkers, together with targeted cancer therapies, are likely to improve drug efficacy and provide a better roadmap for clinical decision making, so that these therapies can be directed to patients who are most likely to benefit. Unfortunately, the biomarkers available for cancer therapy are not sufficient.

One hallmark for tumors is the dysregulation of apoptotic pathways, which play an important role in tumorigenesis. Moreover, resistance to apoptosis is associated with desensitization to conventional cytotoxic and targeted therapies. Thus, the induction of cell death by targeting apoptotic pathways has been an attractive therapeutic strategy.

Two apoptotic pathways are well known. The extrinsic pathway is activated by ligand engagement of cell surface death receptors, and the intrinsic pathway initiates apoptosis by involving a wide-array of non-receptor-mediated stimuli that produce intracellular signals that act directly on targets within the cell and are typically mitochondrial-initiated events.

Several chemotherapeutic agents are known to cause apoptosis. The mechanism often involves changes in the levels and interactions of a key protein family of the intrinsic pathway: the BCL-2 family of proteins. The members of the BCL-2 family are typically designated based on their BCL-2 homology (BH) domains and involvement in apoptosis regulation. Traditionally, these proteins are organized into one of three subfamilies: (1) anti-apoptotic, and (2) BH3-only (pro-apoptotic), or (3) pore-forming or ‘executioner’ (pro-apoptotic) proteins.

To understand the molecular mechanics of individual apoptotic proteins, BH3 profiling is a functional assay that is used to measure tumor cell mitochondrial priming. BH3 profiling measures mitochondrial outer membrane permeabilization (MOMP) following exposure to a peptide-mimicking BH3 domains of BH3-only proteins. MOMP is measured indirectly by the fluorescent dye JC-1, which measures potential across the mitochondrial inner membrane. This potential from the JC-1 dye rapidly degrades in response to MOMP.

However, despite its use for BH3 profiling, the JC-1 readout has several difficulties, which include the inconsistent fluorescent signaling measurements. Further, direct measurement of the protein levels of individual BH3-only proteins, instead of a functional signal, is confounded by the fact that changes in these levels are not consistently correlated with sensitivity to the test anti-cancer agents being tested.

Additionally, combining the functional BH3 measurement with direct measurement of the protein levels of individual BH3-only proteins is complicated and not suited for solid tumor or fixed specimens.

Thus, there is a need for new compositions and methods that provide improved predictive testing for cancer treatment, so that drugs are assigned to patients who are most likely to benefit.

### SUMMARY

Accordingly, the present disclosure is based, in part, on the discovery of several antibodies that each specifically bind to a MCL-1 and BIM heterodimer. The disclosure further provides antibodies are useful for detecting a MCL-1 and BIM heterodimer in a solid tumor or liquid tumor sample from a patient, or a blood cancer sample from a patient, and determining a ratio of the heterodimer to a reference value, the ratio being predictive of a patient's sensitivity to the cancer treatment. As such, the disclosed antibodies provide improved compositions and methods predictive testing for cancer treatment.

In some aspects, the present disclosure provides a composition comprising an antibody or antibody format, or fragment thereof, comprising: (i) a heavy chain variable region comprising heavy chain CDR1, CDR2, and CDR3 sequences, wherein the heavy chain CDR1 sequence is SYAMS (SEQ ID NO: 1), or a variant thereof, the heavy chain CDR2 sequence is TISSGGFATYYPDTVKG (SEQ ID NO: 2), or a variant thereof, and the heavy chain CDR3 sequence is HGGGSYGWFAY (SEQ ID NO: 3), or a variant thereof, and (ii) a light chain variable region comprising light chain CDR1, CDR2, and CDR3 sequences, wherein the light chain CDR1 sequence is ITSTDIDDDMN (SEQ ID NO: 4), or a variant thereof, the light chain CDR2 sequence is EGNTLRP (SEQ ID NO: 5), or a variant thereof, and the light chain CDR3 sequence is LQSDNMPYT (SEQ ID NO: 6), or a variant thereof.

In some embodiments, the antibody or antibody format, or fragment thereof, further comprises variable region framework (FW) sequences juxtaposed between the CDRs according to the formula (FW1)-(CDR1)-(FW2)-(CDR2)-(FW3)-(CDR3)-(FW4), wherein the variable region FW sequences in the heavy chain variable region are heavy chain variable region FW sequences, and wherein the variable region FW sequences in the light chain variable region are light chain variable region FW sequences.

In some embodiments, the variable region FW sequences are human. In some embodiments, the antibody or antibody format, or fragment thereof, comprises a human heavy chain and light chain constant regions. In some embodiments, the constant regions are selected from the group consisting of human IgG1, IgG2, IgG3, and IgG4.

In some embodiments, the antibody or antibody format, or fragment thereof, comprises: (i) a heavy chain variable region sequence comprising the amino acid sequence set forth in SEQ ID NO: 7, or the amino acid sequence of SEQ ID NO: 7 having at least about 90% identity thereto; and (ii) a light chain variable region sequence comprising the amino acid sequence of SEQ ID NO: 8, or the amino acid sequence of SEQ ID NO: 8 having at least about 90% identity thereto. In some embodiments, the antibody or antibody format, or fragment thereof, comprises an amino acid sequence having at least about 95%, or 97%, or 98% identity with SEQ ID NO: 7 and/or SEQ ID NO. 8.

In some embodiments, disclosed herein is a polynucleotide comprising a nucleic acid sequence encoding the antibody or antibody format, or fragment thereof. In some embodiments, disclosed herein is a vector comprising the polynucleotide. In some embodiments, the present disclosure provides a host cell comprising the vector.

In various embodiments, disclosed herein is a pharmaceutical composition comprising the antibody or antibody format, or fragment thereof, and a pharmaceutically acceptable excipient.

In some embodiments, the present disclosure provides a composition a composition comprising an antibody or antibody format, or fragment thereof comprising: (i) a heavy chain variable region comprising heavy chain CDR1, CDR2, and CDR3 sequences, wherein the heavy chain CDR1 sequence is PIAYMS (SEQ ID NO: 9), or a variant thereof, the heavy chain CDR2 sequence is DILPSIGRTIYGEKFED (SEQ ID NO: 10), or a variant thereof, and the heavy chain CDR3 sequence is QDTYYAMDY (SEQ ID NO: 11), or a variant thereof, and (ii) a light chain variable region comprising light chain CDR1, CDR2, and CDR3 sequences, wherein the light chain CDR1 sequence is SASSSVSYMH (SEQ ID NO: 12), or a variant thereof, the light chain CDR2 sequence is STSNLAS (SEQ ID NO: 13), or a variant thereof, and the light chain CDR3 sequence is QQRSSYPYT (SEQ ID NO: 14), or a variant thereof.

In some embodiments, the antibody or antibody format, or fragment thereof, further comprises variable region framework (FW) sequences juxtaposed between the CDRs according to the formula (FW1)-(CDR1)-(FW2)-(CDR2)-(FW3)-(CDR3)-(FW4), wherein the variable region FW sequences in the heavy chain variable region are heavy chain variable region FW sequences, and wherein the variable region FW sequences in the light chain variable region are light chain variable region FW sequences. In some embodiments, the variable region FW sequences are human.

In some embodiments, the antibody or antibody format, or fragment thereof, comprises a human heavy chain and light chain constant regions.

In some embodiments, the constant regions are selected from the group consisting of human IgG1, IgG2, IgG3, and IgG4.

In some embodiments, the present disclosure provides an antibody or antibody format, or fragment thereof, comprises: (i) a heavy chain variable region sequence comprising the amino acid sequence set forth in SEQ ID NO: 15, or the amino acid sequence of SEQ ID NO: 15, or an amino acid sequence having at least about 90% identity thereto; and (ii) a light chain variable region sequence comprising the amino acid sequence of SEQ ID NO: 16, or the amino acid sequence of SEQ ID NO: 16 having at least about 90% identity thereto.

In some embodiments, the antibody or antibody format, or fragment thereof, comprises an amino acid sequence having at least about 95%, or 97%, or 98% identity with SEQ ID NO: 15 and/or SEQ ID NO. 16.

In some embodiments, disclosed herein is a polynucleotide comprising a nucleic acid sequence encoding the antibody or antibody format, or fragment thereof. In some embodiments, disclosed herein is a vector comprising the polynucleotide. In some embodiments, the present disclosure provides a host cell comprising the vector.

In various embodiments, disclosed herein is a pharmaceutical composition comprising the antibody or antibody format, or fragment thereof, and a pharmaceutically acceptable excipient.

In some embodiments, the present disclosure provides a method for predicting a patient's sensitivity or response to a cancer treatment, comprising: (a) contacting a sample with an antibody or antibody format, or fragment thereof, from any one of claims 1-18, wherein the antibody recognizes a heterodimer comprising two B-cell lymphoma 2 (BCL-2) proteins selected from MCL-1 and BIM, the sample being a specimen from a solid tumor or liquid tumor sample of the patient; (b) detecting a signal that indicates the amount of the heterodimer; and (c) determining a ratio of the amount of heterodimer present in the sample from step (b) to a reference value, wherein the reference value comprises the amount of one of the MCL-1 and BIM monomers of the heterodimer in the sample, the ratio being predictive of the patient's sensitivity to the cancer treatment.

In some embodiments, the present disclosure provides a method for predicting a patient's sensitivity or response to a cancer treatment, comprising: (a) contacting a sample with an antibody or antibody format, or fragment thereof, from any one of claims 1-18, wherein the antibody recognizes a heterodimer comprising two B-cell lymphoma 2 (BCL-2) proteins selected from MCL-1 and BIM, and an antibody or antibody format, or fragment thereof, that recognizes one of the MCL-1 and BIM protein monomers of the heterodimer, the sample being a specimen from a solid tumor or liquid tumor of the patient; (b) detecting a signal that indicates the amount of the heterodimer and a signal that indicates the amount of the monomer; and (c) determining a ratio based on the amount heterodimer to the amount of the monomer, the ratio being predictive of the patient's sensitivity to the cancer treatment.

The details of one or more examples of the disclosure are set forth in the description below. Other features or advantages of the present disclosure will be apparent from the following drawings, detailed description of several examples, and also from the appended claims. The details of the disclosure are set forth in the accompanying description below. Although methods and materials similar or equivalent to those described herein can be used in the practice or testing of the present disclosure, illustrative methods and materials are now described. Other features, objects, and advantages of the disclosure will be apparent from the description and from the claims. In the specification and the appended claims, the singular forms also include the plural unless the context clearly dictates otherwise. Unless defined otherwise, all technical and scientific terms used herein have the same meaning as commonly understood by one of ordinary skill in the art to which this disclosure belongs.

### DESCRIPTION OF THE DRAWINGS

FIG. 1 is a graph showing the selective binding of the MCL-1/BIM heterodimer (HSMCB) antibody to the MCL-1/BIM heterodimer (left panel: an antibody having CDRs selected from SEQ ID NO: 9, SEQ ID NO: 10, SEQ ID NO: 11, SEQ ID NO: 12, SEQ ID NO: 13, SEQ ID NO: 14, and having a variable heavy chain selected from SEQ ID NO: 15, and a variable light chain selected from SEQ ID NO: 16), and not to the BIM monomer. The right panel shows the selective binding of the MCL-1/BIM heterodimer (HSMCB) antibody to the MCL-1/BIM heterodimer (right panel: an antibody having CDRs selected from SEQ ID NO: 1, SEQ ID NO: 2, SEQ ID NO: 3, SEQ ID NO: 4, SEQ ID NO: 5, SEQ ID NO: 6, and having a variable heavy chain selected from SEQ ID NO: 7, and a variable light chain selected from SEQ ID NO: 8). In each panel, the top line is the MCL-1/BIM heterodimer, and the bottom lines represent MCL-1 and BSA, respectively.

FIG. 2 is a graph showing the selective binding of the MCL-1/BIM heterodimer (HSMCB) antibody to the MCL-1/BIM heterodimer, and the stability of this antibody at 4° C. for about 2 months. Each line in the graph follows the order in the legend (i.e., the top line is for sample 5-01-20b, the next line is for 5-22-20b, the next line is for 5-29b; this pattern is throughout the entire graph). The antibody in this figure is an antibody having CDRs selected from SEQ ID NO: 9, SEQ ID NO: 10, SEQ ID NO: 11, SEQ ID NO: 12, SEQ ID NO: 13, SEQ ID NO: 14, and having a variable heavy chain selected from SEQ ID NO: 15, and a variable light chain selected from SEQ ID NO: 16.

FIG. 3 shows a set of immunohistochemical (IHC) images relating to the application of the MCL-1/BIM heterodimer antibody (HSMCB) to formalin-fixed paraffin-embedded (FFPE) specimens from MCF-7 breast cancer cells, and HCC70 triple negative breast cancer cells. The MCL-1/BIM heterodimer antibody (HSMCB) requires BIM to bind in situ. BIM siRNA was used on both MCF-7 and HCC70 cells (far left, and right middle columns, respectively), and the cells were then formalin-fixed and paraffin embedded into a tissue microarray (TMA) block, sectioned and added to glass slides, and stained with HSMCB. IHC images of the wild-type (WT) staining for both MCF-7 and HCC70 cells are shown in the middle-left, and far right columns, respectively. The IHC images show a reduced HSMCB IHC signal intensity in BIM-siRNA transfected MCF-7 cells, and BIM-siRNA transfected HCC70 cells. The images across the top panel in FIG. 3 are of an HSMCB antibody having CDRs selected from SEQ ID NO: 9, SEQ ID NO: 10, SEQ ID NO: 11, SEQ ID NO: 12, SEQ ID NO: 13, SEQ ID NO: 14, and having a variable heavy chain selected from SEQ ID NO: 15, and a variable light chain selected from SEQ ID NO: 16; and the images across the bottom panel in FIG. 3 are of an HSMCB antibody having CDRs selected from SEQ ID NO: 1, SEQ ID NO: 2, SEQ ID NO: 3, SEQ ID NO: 4, SEQ ID NO: 5, SEQ ID NO: 6, and having a variable heavy chain selected from SEQ ID NO: 7, and a variable light chain selected from SEQ ID NO: 8.

FIG. 4 shows IHC images comparing the application of HSMCB, and the Bcl-xL/BIM heterodimer antibody (HSBXB), to formalin-fixed paraffin-embedded (FFPE) specimens from HCC70 cells. In the far left panel. IHC images of the HSMCB antibody are shown for both WT HCC70 cells, and Bim-siRNA transfected HCC70 cells. In the middle panel. IHC images of the HSBXB antibody are shown for both WT HCC70 cells, and Bim-siRNA transfected HCC70 cells. In the far right panel. IHC images are shown of HCC70 cells without application of HSMCB or HSBXB. The IHC images show reduced activity for both HSMCB and HSBXB in Bim-siRNA transfected HCC70 cells. The images in the left column of FIG. 4 are of an HSMCB antibody having CDRs selected from SEQ ID NO: 9, SEQ ID NO: 10, SEQ ID NO: 11, SEQ ID NO: 12, SEQ ID NO: 13, SEQ ID NO: 14, and having a variable heavy chain selected from SEQ ID NO: 15, and a variable light chain selected from SEQ ID NO: 16.

FIG. 5 shows IHC images comparing HSMCB and HSBXB reactivity with separate serial sections from the same FFPE patient derived xenotransplant (PDX) to test the performance of Leica and HALO image analysis software for performing segmentation of cancerous cells and the antibody signals within them. The far left panel shows HSMCB staining, the middle panel shows HSBXB staining, and the far right panel shows no antibody staining. The far left image in FIG. 5 is of an HSMCB antibody having CDRs selected from SEQ ID NO: 9, SEQ ID NO: 10, SEQ ID NO: 11, SEQ ID NO: 12, SEQ ID NO: 13, SEQ ID NO: 14, and having a variable heavy chain selected from SEQ ID NO: 15, and a variable light chain selected from SEQ ID NO: 16.

FIG. 6A shows IHC images of PDX Cell Segmentation Performance following Leica image analysis software applied cellular segmentation according to magnitude of cytoplasmic staining. The far left panel shows HSBXB staining, the middle panel shows no antibody staining, and the far right panel shows HSMCB staining. The images on top in each panel are raw images, and the images on the bottom in each panel show the cell cytoplasm segmented and categorized by intensity of staining and color coded. The far right images in FIG. 6A are of an HSMCB antibody having CDRs selected from SEQ ID NO: 9, SEQ ID NO: 10, SEQ ID NO: 11, SEQ ID NO: 12, SEQ ID NO: 13, SEQ ID NO: 14, and having a variable heavy chain selected from SEQ ID NO: 15, and a variable light chain selected from SEQ ID NO: 16. FIG. 6B shows the quantitative Excel output using the Leica software package for the HSBXB staining (far left), no antibody staining (middle), and HSMCB staining (far right).

FIG. 7 shows IHC images of PDX Cell Segmentation Performance (by enlarged nucleus) using HALO image analysis software. The top panel shows no antibody staining (“No Primary”), the middle panel shows HSBXB staining, and the bottom panel shows HSMCB staining. The images in the far left column are raw images, the images in the middle column are selected areas of interest (A.O.I.) images, and the images in the far right column are images following segmentation of individual (presumed) cancerous cells by their respective enlarged nuclei. The images across the bottom row of FIG. 7 are of an HSMCB antibody having CDRs selected from SEQ ID NO: 9, SEQ ID NO: 10, SEQ ID NO: 11, SEQ ID NO: 12, SEQ ID NO: 13, SEQ ID NO: 14, and having a variable heavy chain selected from SEQ ID NO: 15, and a variable light chain selected from SEQ ID NO: 16.

FIG. 8 shows IHC images of HSMCB (left column), and HSBXB staining (right column) with serial sections from a FFPE breast cancer patient biopsy (BCa_01 NV401) using a 10× objective. The HSMCB demonstrates higher reactivity with this tissue sample than HSBXB. The image on the left side of FIG. 8 is of an HSMCB antibody having CDRs selected from SEQ ID NO: 9, SEQ ID NO: 10, SEQ ID NO: 11, SEQ ID NO: 12, SEQ ID NO: 13, SEQ ID NO: 14, and having a variable heavy chain selected from SEQ ID NO: 15, and a variable light chain selected from SEQ ID NO: 16.

FIG. 9 shows IHC images of HSMCB (left column), and HSBXB staining (right column). The samples shown in FIG. 9 are from FIG. 8, and were acquired at a heightened magnification (40× objective) highlighting areas containing enlarged (and positively stained) cancerous cells amongst smaller tumor infiltrating lymphocytes. The image on the left side of FIG. 9 is of an HSMCB antibody having CDRs selected from SEQ ID NO: 9, SEQ ID NO: 10, SEQ ID NO: 11, SEQ ID NO: 12, SEQ ID NO: 13, SEQ ID NO: 14, and having a variable heavy chain selected from SEQ ID NO: 15, and a variable light chain selected from SEQ ID NO: 16.

FIG. 10 shows a schematic (top) of the conditions to immobilize Mcl-1-GST to glutathione coated (GSH) beads. FIG. 10 also shows detection of Mcl-1-GST-GSH beads using an anti-Mcl-1 antibody added at serial dilutions (bottom). In the graph (bottom), the top line is 1 μg/50 μl, the middle line is 0.5 μg/50 μl, and the bottom line is 0.1 μg/50 μl, representing the quantity of recombinant GST-Mcl incubated with 50 μl of GSH beads.

FIG. 11 shows four graphs that estimate the Mcl-1 binding site per bead by Quantibrite beads. A rabbit monoclonal anti-MCL1 antibody from Cell Signaling Technologies was used in this assay. FIG. 11 shows the establishment of bead based system to measure levels of MCL-1 in assaved cells. The top left graph shows the average MFIs of the four different bead populations (coated with different concentrations of GST-Mcl1) following anti-Mell staining. The top right graph is a line graph showing the correlation between the MFI of the beads (x-axis) with the coating concentration (y-axis) to define the linear regression. The bottom left graph uses the linear regression to define the number of antibody binding site per bead, when adding a serial dilution of anti-Mcl1 antibody. The bottom right graph shows how the bead reactivity to anti-Mcl1 can be used to define thresholds of high (High Mcl-1 ctrl) and low (Low Mcl-1 CTRL) levels of Mcl-1, which can be applied to measure the level of Mcl1 in analyzed cells.

FIG. 12 shows a schematic (top) of the conditions to immobilize covalent Mcl-1-Bim complex onto GSH beads, and detection with HSMCB. Mcl-1-Bim bead detection by HSMCB is demonstrated (bottom graph, FIG.12). In the experiments on the left side of the graph for HSMCB (i.e., the first two columns from the left on the graph), the HSMCB antibody is an antibody having CDRs selected from SEQ ID NO: 1, SEQ ID NO: 2, SEQ ID NO: 3, SEQ ID NO: 4, SEQ ID NO: 5, SEQ ID NO: 6, and having a variable heavy chain selected from SEQ ID NO: 7, and a variable light chain selected from SEQ ID NO: 8. In the next experiment near the middle of the graph for HSMCB (i.e., the second set of two columns in the middle on the graph), the antibody is an antibody having CDRs selected from SEQ ID NO: 9, SEQ ID NO: 10, SEQ ID NO: 11, SEQ ID NO: 12, SEQ ID NO: 13, SEQ ID NO: 14, and having a variable heavy chain selected from SEQ ID NO: 15, and a variable light chain selected from SEQ ID NO: 16. The antibody reactive with BcL-XL-Bim is used as a negative control, and Mcl1 (which recognizes both Mcl-1 monomer and Mcl-1-Bim complex) is used as a positive control. The signals using a directly labeled primary antibody (i.e., the first column in each set of two columns throughout the graph) and a secondary antibody detection system (i.e., the second column in each set of two columns throughout the graph) are shown.

FIG. 13 shows a series of flow cytometry graphs each depicting the signal detected by direct labeling of the HSMCB antibody, compared to the signal obtained by two-step labeling of the HSMCB antibody on Mcl-Bim coated beads. FIG. 13 also shows detection of Mcl-1-Bim beads (bottom). The second column in the top panel of FIG. 13 shows an HSMCB antibody having CDRs selected from SEQ ID NO: 1, SEQ ID NO: 2, SEQ ID NO: 3, SEQ ID NO: 4, SEQ ID NO: 5, SEQ ID NO: 6, and having a variable heavy chain selected from SEQ ID NO: 7, and a variable light chain selected from SEQ ID NO: 8, and the third column in top panel of FIG. 13 shows an HSMCB an antibody having CDRs selected from SEQ ID NO: 9, SEQ ID NO: 10, SEQ ID NO: 11, SEQ ID NO: 12, SEQ ID NO: 13, SEQ ID NO: 14, and having a variable heavy chain selected from SEQ ID NO: 15, and a variable light chain selected from SEQ ID NO: 16.

FIG. 14 shows a schematic (top) of the conditions to immobilize HSMCB on Mcl-1-Bim non-covalently bound beads, in an analogous manner to that depicted in FIG. 10. FIG. 14 (bottom) shows a table (bottom, left) of the level of HSMCB when Mcl-1 beads are incubated with Bim. The first and third columns in the table show an antibody having CDRs selected from SEQ ID NO: 1, SEQ ID NO: 2, SEQ ID NO: 3, SEQ ID NO: 4, SEQ ID NO: 5, SEQ ID NO: 6, and having a variable heavy chain selected from SEQ ID NO: 7, and a variable light chain selected from SEQ ID NO: 8, and the second and fourth columns show an antibody having CDRs selected from SEQ ID NO: 9, SEQ ID NO: 10, SEQ ID NO: 11, SEQ ID NO: 12, SEQ ID NO: 13, SEQ ID NO: 14, and having a variable heavy chain selected from SEQ ID NO: 15, and a variable light chain selected from SEQ ID NO: 16. FIG. 14 (bottom) shows a graph (bottom, right) of HSMCB on Mcl-1-Bim non-covalently bound beads. The two top lines in FIG. 14 show an antibody having CDRs selected from SEQ ID NO: 1, SEQ ID NO: 2, SEQ ID NO: 3, SEQ ID NO: 4, SEQ ID NO: 5, SEQ ID NO: 6, and having a variable heavy chain selected from SEQ ID NO: 7, and a variable light chain selected from SEQ ID NO: 8, and the two bottom lines in FIG. 14 show an antibody having CDRs selected from SEQ ID NO: 9, SEQ ID NO: 10, SEQ ID NO: 11, SEQ ID NO: 12, SEQ ID NO: 13, SEQ ID NO: 14, and having a variable heavy chain selected from SEQ ID NO: 15, and a variable light chain selected from SEQ ID NO: 16.

FIG. 15A and FIG. 15B show intracellular flow cytometry measurements in acute myeloid leukemia (“AML”) biopsies. Patient samples were fixed, permeabilized, stained with HSMCB and anti-Mcl-1 antibody, and measured by flow cytometry. HSMCB/Mcl-1 ratios of blast cells were correlated to patient diagnosis data using TIBCO Spotfire software, white lines represent the median value. (FIG. 15A) HSMCB/Mcl-1 ratio was correlated to FLT3/ITD mutation status. FLT3/ITD positivity (marker for poor prognosis in AML) correlated with low HSMCB/Mcl-1 ratio. (FIG. 15B) HSMCB/Mcl-1 ratio was correlated to disease stage. AML patients with refractory and relapse status had lower HSMCB/Mcl-1 ratio compared to newly diagnosed patients. In these experiments, an antibody having CDRs selected from SEQ ID NO: 9, SEQ ID NO: 10, SEQ ID NO: 11, SEQ ID NO: 12, SEQ ID NO: 13, SEQ ID NO: 14, and having a variable heavy chain selected from SEQ ID NO: 15, and a variable light chain selected from SEQ ID NO: 16 was used.

FIG. 16 is a non-limiting schematic overview showing the experimental steps of a method for selecting, isolating and purifying a MCL-1 and BIM heterodimer antibody.

FIG. 17 is a graph showing a BCL2:BIM complex specific antibody that tracts with complex disruption by a BH3 mimetic.

FIG. 18 is a graph showing a ratio of measured heterodimer to monomer in Bcl-xL dependent cell lines: Bcl-2:Bim, Mcl-1:Bim, and Bcl-xL: Bim shifts against Bcl-xL and Mcl-1 when treated.

FIG. 19 is a graph showing how sensitivity to selective BH3 mimetics corresponds to a selection of antibody readouts (“PRIMAB”).

FIG. 20A and FIG. 20B are images showing how BH3-mimetics shift priming signals detected by an alternate method aligns with PRIMAB readout.

### DETAILED DESCRIPTION

The present disclosure is based, in part, on the discovery of compositions and methods for detecting whether a patient is sensitive to a cancer treatment for instance, by an antibody that specifically binds to a MCL-1/BIM heterodimer. The disclosure further provides compositions and methods that are useful for detecting a heterodimer comprising two B-cell lymphoma 2 (BCL-2) proteins selected from MCL-1 and BIM in a solid tumor or liquid tumor sample from a patient, or a blood cancer sample from a patient, and determining a ratio of the heterodimer to a reference value, the ratio being predictive of a patient's sensitivity to the cancer treatment. Importantly, the present methods give information about a cancer patient response based on a direct signal, as opposed to a functional one.

Apoptosis is a process of programmed cell death mediated by a number of signaling pathways that converge at the mitochondria. A group of mitochondrial proteins, i.e., the B cell leukemia/lymphoma-2 (BCL-2) family of proteins, regulates this process. More specifically, pro-apoptotic and anti-apoptotic BCL-2 proteins form heterodimers with their cognate regulating BCL-2 proteins (i.e., the BH3-only BCL-2 proteins), thereby effecting cell death or survival signals.

One of the hallmarks of apoptosis is mitochondrial outer membrane permeabilization (MOMP), a process regulated by the Bcl-2 family of proteins. The activity of this family of proteins is linked to the onset of lymphoid and several solid tumor cancers and is believed in many cancers to be a key mediator of resistance to chemotherapy. Bcl-2 proteins are regulated by distinct protein-protein interactions between pro-survival (anti-apoptotic) and pro-apoptotic members. These interactions occur primarily through BH3 (Bcl-2 homology domain-3) mediated binding. Apoptosis-initiating signaling occurs for the most part upstream of the mitochondria and causes the translocation of short, BH3-only, Bcl-2 family members to the mitochondria where they either activate or sensitize MOMP. The activator BH3 only proteins, Bim and Bid, bind to and directly activate the effector, pro-apoptotic proteins Bax and Bak, and also bind to and inhibit the anti-apoptotic Bcl-2 family proteins, Bcl-2, Mcl-1, Bfl-1, Bcl-w and Bcl-xL. The sensitizer BH3 proteins, Bad, Bik, Noxa, Hrk, Bmf and Puma, bind only to the anti-apoptotic Bcl-2 family proteins. Bcl-2, Mcl-1, Bfl-1, Bcl-w and Bcl-xL, blocking their anti-apoptotic functions. Without wishing to be bound by theory, each sensitizer protein has a unique specificity profile. For example, Noxa (A and B) bind with high affinity to Mcl-1, Bad binds to Bcl-xL and Bcl-2 but only weakly to Mcl-1, and Puma binds well to all three targets. An anti-apoptotic function of these proteins is the sequestering of the activator BH3 protein Bim and Bid by binding to form heterodimers. Displacement of these activators by sensitizer peptides or treatments results in Bax/Bak-mediated apoptotic commitment. These interactions can have various outcomes, including, without limitation, homeostasis, cell death, sensitization to apoptosis, and blockade of apoptosis.

Most effective cancer drugs induce apoptosis in target cancer cells. However, one significant shortfall in current cancer treatment is that different cancer cells can respond to an apoptosis-inducing drug in a variety of manners. This is due, in part, to the presence of different heterodimers between the pro/anti-apoptotic BCL-2 proteins and the regulatory BH3-only BCL-2 proteins in those cancer cells.

In some aspects, the present disclosure provides a method for predicting a patient's sensitivity or response to a cancer treatment, comprising contacting a sample with an antibody or antibody format that recognizes a heterodimer comprising two B-cell lymphoma 2 (BCL-2) proteins selected from MCL-1 and BIM, the sample being a specimen from a solid tumor or liquid tumor of the patient; or a blood cancer sample from a patient, detecting a signal that indicates the amount of the heterodimer; and determining a ratio based on the amount of heterodimer present in the sample to a reference value, wherein the reference value comprises the amount of one of the MCL-1 and BIM protein monomers of the heterodimer in the sample, the ratio being predictive of a patient's sensitivity to the cancer treatment. In some embodiments, the detected signal is from the MCL-1/BIM heterodimeric antibody; for example, detecting a signal indicates the amount of the heterodimer, wherein the signal is from the MCL-1/BIM heterodimeric antibody.

In another aspect, the present disclosure provides a method for predicting a patient's sensitivity or response to a cancer treatment, comprising: contacting a sample with an antibody or antibody format that recognizes a heterodimer comprising two B-cell lymphoma 2 (BCL-2) proteins selected from MCL-1 and BIM and an antibody or antibody format that recognizes one of the MCL-1 and BIM protein monomers of the heterodimer, the sample being a specimen from a solid tumor or liquid tumor of the patient; or a blood cancer sample from a patient, detecting a signal that indicates the amount of the heterodimer and the amount of the monomer; and determining a ratio based on the amount heterodimer to the amount of the monomer, the ratio being predictive of a solid tumor, liquid tumor or a blood cancer patient's sensitivity to the cancer treatment. In some embodiments, the detected signal is from the MCL-1/BIM heterodimeric antibody; for example, detecting a signal indicates the amount of the heterodimer, wherein the signal is from the MCL-1/BIM heterodimeric antibody.

### Generation and Production of Antibodies that Bind to a MCL-1 and BIM Heterodimer and Their Utility in Cancer

The present disclosure can use the determination of a cancer cell's predisposition to undergo apoptosis to elucidate the cancer's susceptibility to a particular treatment. One way this can be done is by using the disclosed antibodies that bind to a Mcl-1 and Bim heterodimer which regulate apoptosis. Formation of a heterodimer induces conformational changes in both members of the heterodimer, resulting in exposure of antigenic epitopes that are sequestered in both members before dimerization. The isolated antibodies of the present disclosure specifically recognize such an epitope and only bind to a Mcl-1 and Bim heterodimer, and not to either non-dimerized member.

One aspect of this disclosure features an isolated antibody that specifically binds to a MCL-1 and BIM heterodimer. The Bcl-2 family includes both Bcl-2 proteins (monomers, i.e., MCL-1 and BIM), and naturally-occurring heterodimers formed of MCL-1 and BIM proteins. The heterodimer contains a first Bcl-2 protein selected from Bim and a second Bcl-2 protein selected from Mcl-1. In some embodiments, the MCL-1 protein in the MCL-1 and Bim heterodimer is associated with Bim, or is associated with the BH3 domain peptide derived from Bim. In some embodiments, the MCL-1 protein in the MCL-1 and BIM heterodimer is associated with multiple different BH3 only proteins, or BH3 peptides derived from them. For example, in some embodiments, the Mcl-1 protein in the Mcl-1 and Bim heterodimer is associated with an activator BH3 protein, and the activator BH3 protein is selected from BID and BIM. In some embodiments, the Mcl-1 protein is associated with a sensitizer BH3 protein. The sensitiver BH3 protein is selected from BAD, BIK, NOXA A, NOXA B, HRK, BMF, and PUMA. In some embodiments, the Mcl-1 protein is associated a multidomain pro-apoptotic protein, and the multidomain pro-apoptotic protein is selected from BAX and BAK.

The methods of the present disclosure also provide a ratio of the MCL-1 and BIM heterodimer to one of a MCL-1 and BIM monomer.

The compositions of the present disclosure include an antibody or antibody format, or fragment thereof, that recognizes a Mcl-1/Bim heterodimer (HSMCB). In various embodiments, the antibody is a full-length multimeric protein that includes two heavy chains and two light chains. Each heavy chain includes one variable region (e.g., VH) and at least three constant regions (e.g., CH₁, CH₂ and CH₃), and each light chain includes one variable region (VL) and one constant region (CL). The variable regions determine the specificity of the antibody. Each variable region comprises three hypervariable regions also known as complementarity determining regions (CDRs) flanked by four relatively conserved framework regions (FRs). The three CDRs, referred to as CDR1, CDR2, and CDR3, contribute to the antibody binding specificity. In some embodiments, the antibody is a chimeric antibody. In some embodiments, the antibody is a humanized antibody.

In some embodiments, the antibody or antibody format, or fragment thereof, is a single-domain antibody, a recombinant heavy-chain-only antibody (VHH), a single-chain antibody (scFv), a shark heavy-chain-only antibody (VNAR), a microprotein (cysteine knot protein, knottin), a DARPin; a Tetranectin; an Affibody; a Transbody; an Anticalin; an AdNectin; an Affilin; a Microbody; a peptide aptamer; an alterase; a plastic antibody; a phylomer; a stradobody; a maxibody; an evibody; a fynomer, an armadillo repeat protein, a Kunitz domain, an avimer, an atrimer, a probody, an immunobody, a triomab, a troybody; a pepbody; a vaccibody, a UniBody; Affimers, a DuoBody, a Fv, a Fab, a Fab′, a F(ab′)2, a peptide mimetic molecule, or a synthetic molecule, as described in US Patent Nos. or Patent Publication Nos. U.S. Pat. No. 7,417,130, US 2004/132094, U.S. Pat. No. 5,831,012, US 2004/023334, U.S. Pat. Nos. 7,250,297, 6,818,418, US 2004/209243, U.S. Pat. Nos. 7,838,629, 7,186,524, 6,004,746, 5,475,096, US 2004/146938, US 2004/157209, U.S. Pat. Nos. 6,994,982, 6,794,144, US 2010/239633, U.S. Pat. No. 7,803,907, US 2010/119446, and/or U.S. Pat. No. 7,166,697, the contents of which are hereby incorporated by reference in their entireties. See also, Storz MAbs. 2011 May-Jun; 3(3): 310-317.

In various embodiments, the antibody or antibody format, or fragment thereof, is a single-domain antibody, such as VHH from, for example, an organism that produces VHH antibody such as a camelid, a shark, or a designed VHH. VHHs are antibody-derived therapeutic proteins that contain the unique structural and functional properties of naturally-occurring heavy-chain antibodies. VHH technology is based on fully functional antibodies from camelids that lack light chains. These heavy-chain antibodies contain a single variable domain (VHH) and two constant domains (CH₂ and CH₃). VHHs are commercially available under the trademark of NANOBODIES.

In some embodiments, the VHH is a humanized VHH or camelized VHH.

In some embodiments, the antibody or antibody format, or fragment thereof is selected from one or more of a monoclonal antibody, polyclonal antibody, Fab, Fab′, Fab′-SH, F(ab′)2, Fv, single chain Fv, diabody, linear antibody, bispecific antibody, multispecific antibody, chimeric antibody, humanized antibody, human antibody, and a fusion protein comprising the antigen-binding portion of an antibody.

In some embodiments, the compositions of the present disclosure include an antibody or antibody format, or fragment thereof, comprising: (i) a heavy chain variable region comprising heavy chain CDR1, CDR2, and CDR3 sequences, wherein the heavy chain CDR1 sequence is SYAMS (SEQ ID NO: 1), or a variant thereof, the heavy chain CDR2 sequence is TISSGGFATYYPDTVKG (SEQ ID NO: 2), or a variant thereof; and the heavy chain CDR3 sequence is HGGGSYGWFAY (SEQ ID NO: 3), or a variant thereof; and (ii) a light chain variable region comprising light chain CDR1, CDR2, and CDR3 sequences, wherein the light chain CDR1 sequence is ITSTDIDDDMN (SEQ ID NO: 4), or a variant thereof, the light chain CDR2 sequence is EGNTLRP (SEQ ID NO: 5), or a variant thereof, and the light chain CDR3 sequence is LQSDNMPYT (SEQ ID NO: 6), or a variant thereof.

In various embodiments, the antibody or antibody format, or fragment thereof, or variant thereof, may comprise an amino acid sequence having one or more amino acid mutations (e.g., substitutions or deletions) relative to any of the sequences disclosed herein. In some embodiments, the one or more amino acid mutations may be independently selected from substitutions, insertions, deletions, and truncations. In embodiments, the antibody or antibody format, or fragment thereof, comprises a sequence that has about 1, 2, 3, 4, 5, 6, 7, 8, 9, or 10 or more amino acid mutations with respect to any one of the amino acid sequences disclosed herein.

In various embodiments, the antibody or antibody format, or fragment thereof, can comprise: (i) a heavy chain variable region sequence comprising the amino acid sequence set forth in SEQ ID NO: 7, or the amino acid sequence of SEQ ID NO: 7 having at least about 90% identity thereto; and (ii) a light chain variable region sequence comprising the amino acid sequence of SEQ ID NO: 8, or the amino acid sequence of SEQ ID NO: 8 having at least about 90% identity thereto. The antibody or antibody format can comprise an amino acid sequence having at least about 93%, or 95%, or 97%, or 98% identity with SEQ ID NO: 7 and/or SEQ ID NO. 8.

In various embodiments, an antibody or antibody format, or fragment thereof, comprises at least one heavy chain of the following amino acid sequence:

(SEQ ID NO: 7)
MNFGLSLIFLVLVLKGVQCEVOLVESGGDLVKPGGSLKLSCAVSGFTF
SSYAMSWVRQTPAKRLEWVATISSGGFATYYPDTVKGRFTISRDNAK
NTLYLQMSSLRSEDTAMYYCARHGGGSYGWFAYWGQGTLVTVSE

In various embodiments, an antibody or antibody format, or fragment thereof, comprises at least one heavy chain of the following nucleotide sequence:

(SEQ ID NO: 17)
ATGAACTTCGGGCTCAGCTTGATTTTCCTTGTCCTTGTTTTAAAAGG
TGTCCAGTGTGAAGTGCAGCTGGTGGAGTCTGGGGGAGACTTAGTG
AAGCCTGGAGGGTCCCTGAAACTCTCCTGTGCAGTTTCTGGATTCA
CTTTCAGTAGCTATGCCATGTCTTGGGTTCGCCAGACTCCGGCGAA
GAGGCTGGAGTGGGTCGCAACCATTAGTAGTGGTGGTTTTGCCACC
TACTATCCAGACACTGTGAAGGGCCGATTCACCATCTCCAGAGACA
ATGCCAAAAACACCCTGTACCTGCAAATGAGCAGTCTGAGGTCTGA
GGACACAGCCATGTATTACTGTGCAAGACATGGGGGTGGTAGCTA
CGGCTGGTTTGCTTACTGGGGCCAAGGGACTCTGGTCACTGTCTCT
GAA

In various embodiments, an antibody or antibody format, or fragment thereof, comprises at least one light chain of the following amino acid sequence:

(SEQ ID NO: 8)
MLSLAPLLSLLLLCVSDSRAETTVTQSPASLSVATGEKVTIRCITSTDI
DDDMNWYQQKPGEPPKLLISEGNTLRPGVPSRFSSSGYGTDFVFTIENT
LSEDFADYYCLQSDNMPYTFGGGTKLEIK

In various embodiments, an antibody or antibody format, or fragment thereof, comprises at least one light chain of the following nucleotide sequence:

(SEQ ID NO: 18)
ATGCTCTCACTAGCTCCTCTCCTCAGCCTTCTTCTCCTCTGTGTCTCT
GATTCTAGGGCAGAAACAACTGTGACCCAGTCTCCAGCATCCCTGT
CCGTGGCTACAGGAGAAAAAGTCACTATCAGATGCATAACCAGCA
CTGATATTGATGATGATATGAACTGGTACCAGCAGAAGCCAGGGG
AACCTCCTAAGCTCCTTATTTCAGAAGGCAATACTCTTCGTCCTGG
AGTCCCATCCCGATTCTCCAGCAGTGGCTATGGCACAGATTTTGTTT
TTACAATTGAAAACACGCTCTCAGAAGATTTTGCAGATTACTACTG
TTTGCAAAGTGATAACATGCCGTACACGTTCGGAGGGGGGACCAA
GCTGGAAATAAAA

In various embodiments, the compositions of the present disclosure include an antibody or antibody format, or fragment thereof, comprising: (i) a heavy chain variable region comprising heavy chain CDR1, CDR2, and CDR3 sequences, wherein the heavy chain CDR1 sequence is PIAYMS (SEQ ID NO: 9), or a variant thereof, the heavy chain CDR2 sequence is DILPSIGRTIYGEKFED (SEQ ID NO: 10), or a variant thereof, and the heavy chain CDR3 sequence is QDTYYAMDY (SEQ ID NO: 11), or a variant thereof, and (ii) a light chain variable region comprising light chain CDR1, CDR2, and CDR3 sequences, wherein the light chain CDR1 sequence is SASSSVSYMH (SEQ ID NO: 12), or a variant thereof, the light chain CDR2 sequence is STSNLAS (SEQ ID NO: 13), or a variant thereof, and the light chain CDR3 sequence is QQRSSYPYT (SEQ ID NO: 14), or a variant thereof.

In various embodiments, the antibody or antibody format, or fragment thereof, or variant thereof, may comprise an amino acid sequence having one or more amino acid mutations (e.g., substitutions or deletions) relative to any of the sequences disclosed herein. In some embodiments, the one or more amino acid mutations may be independently selected from substitutions, insertions, deletions, and truncations. In embodiments, the antibody or antibody format, or fragment thereof, comprises a sequence that has about 1, 2, 3, 4, 5, 6, 7, 8, 9, or 10 or more amino acid mutations with respect to any one of the amino acid sequences disclosed herein.

In some embodiments, the antibody or antibody format, or fragment thereof, can comprise: (i) a heavy chain variable region sequence comprising the amino acid sequence set forth in SEQ ID NO: 15, or the amino acid sequence of SEQ ID NO: 15, or an amino acid sequence having at least about 90% identity thereto; and (ii) a light chain variable region sequence comprising the amino acid sequence of SEQ ID NO: 16, or the amino acid sequence of SEQ ID NO: 16 having at least about 90% identity thereto. The antibody or antibody format can comprise an amino acid sequence having at least about 93%, or 95%, or 97%, or 98% identity with SEQ ID NO: 15 and/or SEQ ID NO. 16.

In various embodiments, an antibody or antibody format, or fragment thereof, comprises at least one heavy chain of the following amino acid sequence:

(SEQ ID NO: 15)
MDWIWIMLHLLAAATGLQSQVHLQQSGSELRSPGSSVKLSCKDFDSE
VFPIAYMSWVRQKPGHGFEWIGDILPSIGRTIYGEKFEDKATLDADTVS
NTAYLELNSLTSEDSAIYYCARQDTYYAMDYWGQGTSVTVSS

In various embodiments, an antibody or antibody format, or fragment thereof, comprises at least one heavy chain of the following nucleotide sequence:

(SEQ ID NO: 19)
ATGGACTGGATTTGGATCATGCTCCATCTGCTGGCAGCAGCTACAG
GTCTCCAATCCCAGGTTCACCTACAACAGTCTGGTTCTGAACTGAG
GAGTCCTGGGTCTTCAGTAAAGCTTTCATGCAAGGATTTTGATTCA
GAAGTCTTCCCTATTGCTTATATGAGTTGGGTTAGGCAGAAGCCTG
GACATGGATTTGAGTGGATTGGTGACATACTCCCAAGTATTGGTAG
AACAATCTATGGAGAGAAGTTTGAGGACAAAGCCACACTGGATGC
AGACACAGTGTCCAACACAGCCTACTTGGAGCTCAACAGTCTGACA
TCTGAGGACTCTGCTATCTACTACTGTGCAAGACAAGACACTTACT
ATGCTATGGACTACTGGGGTCAAGGAACCTCAGTCACCGTCTCCTC
A

In various embodiments, an antibody or antibody format, or fragment thereof, comprises at least one light chain of the following amino acid sequence:

(SEQ ID NO: 16)
MHFQVQIFSFLLISASVIMSRGQIVLTQSPAIMSASPGEKVTITCSASSS
VSYMHWVQQKPGTSPKLWIYSTSNLASGVPARFRGSGSGTSYSLTISRM
EAEDAATYYCQQRSSYPYTFGGGTKLEIK

In various embodiments, an antibody or antibody format, or fragment thereof, comprises at least one light chain of the following nucleotide sequence:

(SEQ ID NO: 20)
ATGCATTTTCAAGTGCAGATTTTCAGCTTCCTGCTAATCAGTGCCTC
AGTCATAATGTCCAGAGGACAAATTGTTCTCACCCAGTCTCCAGCA
ATCATGTCTGCATCTCCAGGGGAGAAGGTCACCATAACCTGCAGTG
CCAGCTCAAGTGTAAGTTACATGCACTGGGTCCAGCAGAAGCCAG
GCACTTCTCCCAAACTCTGGATTTATAGCACATCCAACCTGGCTTCT
GGAGTCCCTGCTCGCTTCAGAGGCAGTGGATCTGGGACCTCTTACT
CTCTCACAATCAGCCGAATGGAGGCTGAAGATGCTGCCACTTATTA
CTGCCAGCAAAGGAGTAGTTACCCGTACACGTTCGGAGGGGGGAC
CAAGCTGGAAATAAAA

In embodiments, the amino acid mutations are amino acid substitutions, and may include conservative and/or non-conservative substitutions.

“Conservative substitutions” may be made, for instance, on the basis of similarity in polarity, charge, size, solubility, hydrophobicity, hydrophilicity, and/or the amphipathic nature of the amino acid residues involved. The 20 naturally occurring amino acids can be grouped into the following six standard amino acid groups: (1) hydrophobic: Met, Ala, Val, Leu, Ile; (2) neutral hydrophilic: Cys, Ser, Thr; Asn, Gln; (3) acidic: Asp, Glu; (4) basic: His, Lys, Arg; (5) residues that influence chain orientation: Gly, Pro; and (6) aromatic: Trp, Tyr, Phe.

As used herein, “conservative substitutions” are defined as exchanges of an amino acid by another amino acid listed within the same group of the six standard amino acid groups shown above. For example, the exchange of Asp by Glu retains one negative charge in the so modified polypeptide. In addition, glycine and proline may be substituted for one another based on their ability to disrupt α-helices.

As used herein, “non-conservative substitutions” are defined as exchanges of an amino acid by another amino acid listed in a different group of the six standard amino acid groups (1) to (6) shown above.

Bcl-2 proteins, which are present in mitochondria, are major regulators of the commitment to programmed cell death and executioners of death/survival signals. (See, e.g., Reed, Natural Clinical Practice Oncology, 3:388-398 (2006), Green et al., Cancer Cell 1:19-30 (2002), and Adams et al., Cold Spring Harb. Symp. Quant. Biol. 70:469-477 (2005)) There are four sub-groups of Bcl-2 proteins: (i) multi-domain anti-apoptotic Bcl-2 proteins, (ii) multi-domain pro-apoptotic Bcl-2 proteins, (iii) activator BH3-only Bcl-2 proteins, and (iv) sensitizer BH3-only Bcl-2 proteins. Table 1 below lists major human Bcl-2 proteins and their GenBank accession numbers:

TABLE 1
Human Bcl-2 Proteins

| Bcl-2 Proteins Class | Bcl-2 proteins | Gene Bank Accession numbers |
|---|---|---|
| Multi Domain | Bcl-2 | AAH27258(Jul. 15, 2006( |
| Anti-Apoptotic | Mcl-1 | AAF64255 (Jul. 15, 2000) |
| | BCL-XL | AAH19307 (Jul. 15, 2015) |
| | BFL-1 | Q16548 (Mar. 3, 2009) |
| | BFL-W | AAB09055 (Sep. 29, 1996) |
| Multi Domain | BAX | Q07812 (Apr. 14, 2009) |
| Pro-Apoptotic | BAK | Q16611 (Apr. 14, 2009) |
| Activator | BID | P55957 (Mar. 3, 2009) |
| BH3 only class | BIM | O43521 (Apr. 14, 2009) |
| Sensitisor | BAD | CAG30276 (Jun. 29, 2004) |
| BH3 only class | NOXA | Q13794 (Mar. 3, 2009) |
| | PUMA. | Q9BXH1 (Apr. 14, 2009) |
| | HRK, | AAC34931 (Sep. 9, 1998) |
| | Mule | Q7Z67Z (Apr. 14, 2009) |
| | BIK | CAG30276 (Oct. 16, 2008) |
| | BMF | AAH63928 (Aug. 19^{th}, 2004) |

Other Bcl-2 proteins, if any, can be identified by a homologous search using the amino acid sequence of a known Bcl-2 protein as a query.

Polypeptides can be identified based on homology to the BH3 domain, and polypeptides can possess at least about 50%, about 60%, about 70%, about 80%, about 90%, about 95%, about 96%, about 97%, about 98%, about 99% sequence homology to the amino acid sequences of the polypeptides disclosed in Table 1. Preferred variants are those that have conservative amino acid substitutions made at one or more predicted non-essential amino acid residues. For example, a “conservative amino acid substitution” is one in which the amino acid residue is replaced with an amino acid residue having a similar side chain. In a further embodiment, the BH3 domain peptide is an activator or a sensitizer of apoptosis. In a preferred embodiment, the BH3 domain peptide is a sensitizer.

If a cell is pre-set to undergo drug-induced apoptosis (e.g. the cell is dependent on Bcl-2 polypeptide activity for survival), the MCL-1 and BIM heterodimer antibodies of the present disclosure can be used to identify the specific MCL-1 and BIM proteins that are responsible for apoptotic block.

It is known that members in one subgroup of Bcl-2 proteins form heterodimers with members in a different subgroup to regulate apoptosis. Formation of a heterodimer induces conformational changes in both members of the heterodimer, resulting in exposure of antigenic epitopes that are sequestered in both members before dimerization. The isolated MCL-1 and BIM heterodimer antibodies of the present disclosure specifically recognize such an epitope. In other words, the antibodies disclosed herein can specifically bind to a heterodimer of the Bcl-2 family selected from MCL-1 and BIM.

Briefly, without wishing to be bound by theory, as a result of aberrant phenotypes. cancer cells develop blocks in apoptosis pathways. These blocks make cancer cells both resistant to some therapies, and, surprisingly, make some cancer cells sensitive to other therapies. The concept of “oncogene addiction” describes the phenomena of the acquired dependence of cancer cells on, or addiction to, particular proteins for survival. Cancer cells can be, but are not always, pre-set to undergo apoptosis and this is a function of these cells being dependent on any, or all of the anti-apoptotic Bcl-2 family proteins for their otherwise unintended survival. This provides insight into the likelihood of a cancer cell to respond to treatment.

Cancer cells, without wishing to be bound by theory, exhibit abnormalities, such as DNA damage, genetic instability, abnormal growth factor signaling, and abnormal or missing matrix interactions, any of which should typically induce apoptosis through the intrinsic (mitochondrial) apoptosis pathway. However, rather than respond to these apoptosis signals cancer cells survive. Often, in doing so, these cells become highly dependent on selected blocks to chronic apoptosis signals. This adaptation provides a survival mechanism for the cancer cells; however, these adaptations can also make cancer cells susceptible to particular apoptosis inducing therapies. A crucial event that commits a cell to die by intrinsic apoptosis is the permeabilization of the mitochondrial outer membrane (MOMP) and the release of molecules that activate the effector caspases. In many cases, MOMP is the point of no return in the intrinsic apoptosis pathway. The Bcl-2 family proteins are the key regulators of MOMP, and their activity is linked to the onset of lymphoid and several solid tumor cancers, and is believed in many cancers to be the key mediator of resistance to chemotherapy.

Bcl-2 proteins are regulated by distinct protein-protein interactions between pro-survival (anti-apoptotic) and pro-apoptotic members. These interactions occur primarily through BH3 (Bcl-2 homology domain-3) mediated binding. Apoptosis-initiating signaling occurs for the most part upstream of the mitochondria and causes the translocation of short, BH3-only, Bcl-2 family members to the mitochondria where they either activate or sensitize MOMP. The activator BH3 only proteins, Bim and Bid, bind to and directly activate the effector, pro-apoptotic proteins Bax and Bak, and also bind to and inhibit the anti-apoptotic Bcl-2 family proteins, Bcl-2, Mcl-1, Bfl-1, Bcl-w and Bcl-xL. The sensitizer BH3 proteins, Bad, Bik, Noxa, Hrk, Bmf and Puma, bind only to the anti-apoptotic Bcl-2 family proteins, Bcl-2, Mcl-1, Bfl-1, Bcl-w and Bcl-xL, blocking their anti-apoptotic functions. Without wishing to be bound by theory, each sensitizer protein has a unique specificity profile. For example, Noxa (A and B) bind with high affinity to Mcl-1, Bad binds to Bcl-xL and Bcl-2 but only weakly to Mcl-1, and Puma binds well to all three targets. An anti-apoptotic function of these proteins is the sequestering of the activator BH3 protein Bim and Bid. Displacement of these activators by sensitizer peptides results in Bax/Bak-mediated apoptotic commitment. These interactions can have various outcomes, including, without limitation, homeostasis, cell death, sensitization to apoptosis, and blockade of apoptosis.

A defining feature of cancer cells in which apoptotic signaling is blocked is an accumulation of the BH3 only activator proteins at the mitochondrial surface, a result of these proteins being sequestered by the anti-apoptotic proteins. This accumulation and proximity to their effector target proteins accounts for increased sensitivity to antagonism of Bcl-2 family proteins in the “BH3 primed” state.

In some embodiments, a cell yielding a high apoptotic response to Noxa (A or B) is Mcl-1 primed. In some embodiments, Puma reflects pan-Bcl-2 family priming. In this way, cells that are dependent on either Mcl-1 or Bim, or both proteins, or on several Bcl-2 family members are readily distinguished so that appropriate treatment may be tailored accordingly. The distinctions in mitochondrial response to these peptides guides the use of therapies that are known to work through pathways that funnel into either Mcl-1 or Bim affected intrinsic signaling. The use of a Mcl-1 inhibiting compound may be indicated in such cases. In some embodiments, the present methods also indicate or contraindicate therapies that target entities upstream of Mcl-1 or Bim.

The antibodies of the present disclosure can be a whole immunoglobulin or a fragment thereof that retains antigen-binding activity. In some embodiments, the antibodies of the present disclosure can be a genetically modified immunoglobulin, including scFv antibody, chimeric antibody, or a humanized antibody. In some embodiments, the antibody or antibody format is selected from one or more of a monoclonal antibody, polyclonal antibody, antibody fragment, Fab, Fab′, Fab′-SH, F(ab′)2, Fv, single chain Fv, diabody, linear antibody, bispecific antibody, multispecific antibody, chimeric antibody, humanized antibody, human antibody, and a fusion protein comprising the antigen-binding portion of an antibody. In some embodiments, the antibody or antibody format further comprises variable region framework (FW) sequences juxtaposed between the CDRs according to the formula (FW1)-(CDR1)-(FW2)-(CDR2)-(FW3)-(CDR3)-(FW4), wherein the variable region FW sequences in the heavy chain variable region are heavy chain variable region FW sequences, and wherein the variable region FW sequences in the light chain variable region are light chain variable region FW sequences. In some embodiments, the variable region FW sequences are human. The antibody or antibody format can further comprise a human heavy chain and light chain constant regions. In some embodiments, the constant regions are selected from the group consisting of human IgG1, IgG2, IgG3, and IgG4. The term “isolated antibody,” as used herein, refers to an antibody substantially free from naturally associated molecules, i.e., the naturally associated molecules constituting at most 20% by dry weight of a preparation containing the antibody.

The antibodies of the present disclosure may be prepared by conventional methods. (See, e.g., Harlow and Lane, (1988) Antibodies: A Laboratory Manual, Cold Spring Harbor Laboratory, New York) For example, a synthetic heterodimer of the Bcl-2 family may be prepared by producing two members of the heterodimer protein separately using a section of a key binding motif, followed by synthesizing the key epitope and inducing a portion of one member of the heterodimer, the ligand, and the full-length protein of the other member of the heterodimer, the receptor. The functionality of the synthetic heterodimer can be checked using in vitro binding assays. Once determined, the binding fidelity is maintained in the synthetic heterodimer, and then the ligand portion can be modified to contain a benzoyl phenylalanine (Anaspec, Fremont, CA, USA) in place of one of several potential aromatic amino acids. Each protein fragment can be further tested for binding fidelity as detailed above. Once selected, the binding ligand can be covalently attached by exposure to activating exposure to UV light at 450 nM for up to 8 hours. The synthetic heterodimer can then be purified by FPLC and be used as an immunogen for injection into a mouse host.

To produce antibodies that bind to the heterodimer, the heterodimer may be optionally coupled to a carrier protein (e.g., KLH) and mixed with an adjuvant, followed by injection into a host animal. Antibodies produced in the animal can then be purified by heterodimer affinity chromatography. Commonly employed host animals include rabbits, mice, guinea pigs, and rats. Various adjuvants may be used to increase the immunological response, which depends on the host species and include Freund's adjuvant (complete and incomplete), mineral gels such as aluminum hydroxide, CpG, surface-active substances such as lysolecithin, pluronic polyols, polyanions, peptides, oil emulsions, keyhole limpet hemocyanin, and dinitrophenol. Useful human adjuvants include BCG (bacille Calmette-Guerin) and Corynebacterium parvum. Polyclonal antibodies, i.e., heterogeneous populations of antibody molecules, are present in the sera of the immunized animal.

Monoclonal antibodies, i.e., homogeneous populations of antibody molecules, are prepared using standard hy bridoma technology. (See, for example, Kohler et al. (1975) Nature 256, 495; Kohler et al. (1976) Eur. J. Immunol. 6, 511; Kohler et al. (1976) Eur J Immunol 6, 292; and Hammerling et al. (1981) Monoclonal Antibodies and T Cell Hybridomas, Elsevier, N.Y.)) In particular, monoclonal antibodies may be obtained by any technique that provides for the production of antibody molecules by continuous cell lines in culture. (See, Kohler et al. (1975) Nature 256, 495; Kosbor et al. (1983) Immunol Today 4, 72; Cole et al. (1983) Proc. Natl. Acad. Sci. USA 80, 2026, and the EBV-hybridoma technique (Cole et al. (1983); see also Monoclonal Antibodies and Cancer Therapy, Alan R. Liss, Inc., pp. 77-96)) Such antibodies can be of any immunoglobulin class including IgG, IgM, IgE, IgA, IgD, and any subclass thereof. The hybridoma producing the monoclonal antibodies of the present disclosure may be cultivated in vitro or in vivo. The ability to produce high titers of monoclonal antibodies in vivo makes it a particularly useful method of production.

In addition, techniques developed for the production of “chimeric antibodies” can be used. (See, e.g., Morrison et al. (1984) Proc. Natl. Acad. Sci. USA 81, 6851; Neuberger et al. (1984) Nature 312, 604; and Takeda et al. (1984) Nature 314:452) A chimeric antibody is a molecule in which different portions are derived from different animal species, such as those having a variable region derived from a murine monoclonal antibody and a human immunoglobulin constant region. Alternatively, techniques described for the production of single chain antibodies (U.S. Pat. Nos. 4,946,778 and 4,704,692) can be adapted to produce a phage or yeast library of scFv antibodies, scFv antibodies are formed by linking the heavy and light chain fragments of the Fv region via an amino acid bridge.

Moreover, antibody fragments can be generated by known techniques. For example, such fragments include, but are not limited to, F(ab′)sub.2 fragments that can be produced by pepsin digestion of an antibody molecule, and Fab fragments that can be generated by reducing the disulfide bridges of F(ab′)sub.2 fragments. Antibodies can also be humanized by methods known in the art. For example, monoclonal antibodies with a desired binding specificity can be commercially humanized (Scotgene, Scotland; and Oxford Molecular, Palo Alto, Calif.). Fully human antibodies, such as those expressed in transgenic animals are also features of the present disclosure (see, e.g., Green et al. (1994) Nature Genetics 7, 13; and U.S. Pat. Nos. 5,545,806 and 5,569,825).

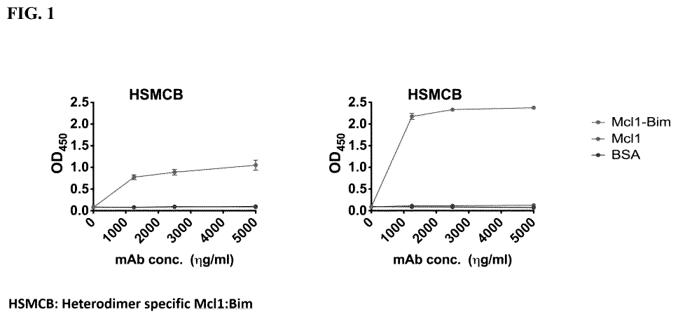

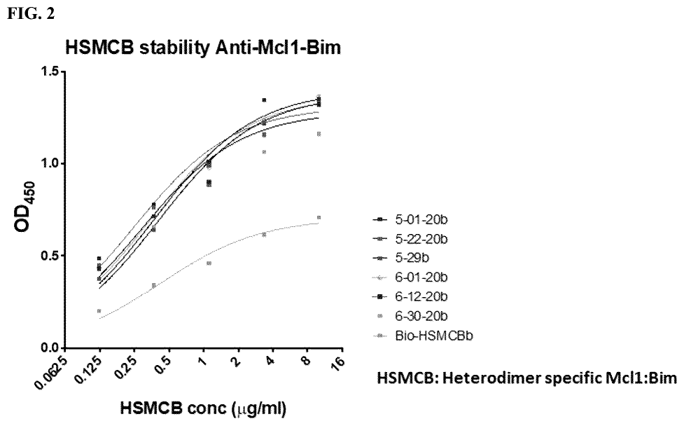

The antibodies prepared by any of the methods described above were confirmed for their binding to a Bcl-2 heterodimer selected from MCL-1 and BIM. (i.e., see FIG. 1 and FIG. 2) They were further subjected to a negative selection to exclude those that also were bound to a non-dimerized member of the heterodimer. In these experiments, negative selection was the unbound MCL-1 protein or the unbound BIM protein. For example, each of the two members, i.e., monomer A and monomer B, may be labeled with a distinct fluorescent dye, i.e., dye x and dye y, respectively. Dyes x and y have different optimal emission wavelengths. The antibody was first incubated with labeled monomer A, labeled monomer B, or the A/B heterodimer (double labeled) for a suitable period and then captured by GamaBind Sepharose beads. Whether the antibody was capable of binding to either monomer or to the heterodimer was determined based on the fluorescent signal released from the captured antibody. Antibodies that bound to the heterodimer and not to either non-dimerized member were selected.

Antibodies were also prepared by any of the methods described above were confirmed for their binding to a Bcl-2 heterodimer selected from MCL-1 and BIM using an ELISA assay.

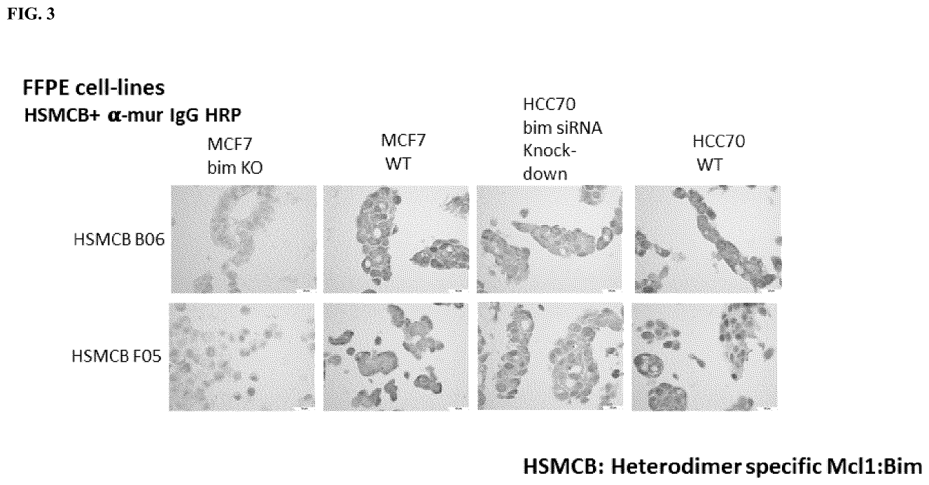

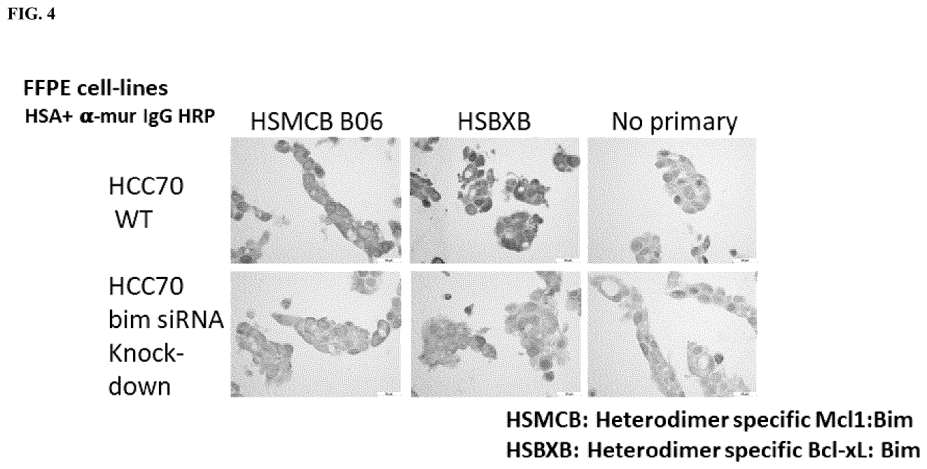

The antibodies disclosed herein can be used in a method of detecting the presence or absence of a Bcl-2 heterodimer in a sample obtained from a subject (e.g., a patient), particularly, a fixed tissue sample or a mitochondrial fraction, by conventional methods, e.g., immunohistochemistry (IHC) staining (i.e., see FIG. 3 and FIG. 4). For example, an antibody that recognizes a heterodimer comprising two B-cell lymphoma 2 (BCL-2) proteins selected from MCL-1 and BIM may be used to profile the presence of MCL-1 and BIM on the outer membranes of mitochondria in multiple tissue samples from a patient. Tissues at various disease stages (e.g., malignancy stages) can be collected from the same patient. Mitochondrial fractions can be prepared from these tissues and, using a plurality of the antibodies of the present disclosure, the fractions can be profiled for the presence/absence of Bcl-2 heterodimers selected from MCL-1 and BIM.

Also disclosed herein is a method of predicting a human patient's sensitivity or responsiveness to a drug that interferes with formation of a particular Bcl-2 heterodimer selected from MCL-1 and BIM, directly or indirectly, based on the presence of the MCL-1 and BIM heterodimer in the patient.

It is well known that Bcl-2 proteins play an essential role in regulating apoptosis via formation of heterodimers between members in different Bcl-2 sub-groups. See Table 1 above. An activator BH3-only Bcl-2 protein (i.e., BID or BIM) binds to a multi-domain pro-apoptotic Bcl-2 protein (i.e., BAX or BAK), triggering mitochondrial outer membrane permeabilization (MOMP), which leads to cell death. A multi-domain anti-apoptotic Bcl-2 protein (e.g., Bcl-2 or Mcl-1) can bind to BAX and BAK, and also sequester an activator BH3-only protein from binding to BAX or BAK. Consequently, it blocks the MOMP process, resulting in cell survival. The activity of a multi-domain anti-apoptotic Bcl-2 protein is regulated by the sensitizer BH3-only proteins. This subgroup of Bcl-2 proteins promotes apoptosis by binding to the anti-apoptotic Bcl-2 protein, displacing the activator BH3-only Bcl-2 proteins so that they are released to bind to the pro-apoptotic Bcl-2 proteins, thereby triggering the MOMP process. In short, there are two types of Bcl-2 heterodimers: (1) pro-apoptotic Bcl-2 heterodimers, formed between an activator BH3-only Bcl-2 protein and a multi-domain pro-apoptotic Bcl-2 protein or between a sensitizer BH3-only Bcl-2 protein and a multi-domain anti-apoptotic Bcl-2 protein; and (2) anti-apoptotic Bcl-2 heterodimers, formed between a multi-domain anti-apoptotic Bcl-2 protein and an activator BH3-only Bcl-2 protein or between a multi-domain anti-apoptotic Bcl-2 protein and a multi-domain pro-apoptotic Bcl-2 protein. Formation of pro-apoptotic Bcl-2 heterodimers promotes apoptosis while formation of anti-apoptotic heterodimers promotes cell survival.

The presence of a particular pro-or anti-apoptotic Bcl-2 heterodimer in a subject (e.g., a patient) is known to indicate that patient's responsiveness to a drug that blocks formation of the particular heterodimer and inhibits its function. (See, e.g., Delbridge and Strasser A. Cell Death Differ. 2015 Jul; 22(7):1071-80, doi: 10.1038/cdd.2015.50)

In some embodiments of the present disclosure, the drug is a mimetic of a BH3-only protein that competes against the BH3-only protein for binding to its cognate partner. In other embodiments, the drug targets an upstream apoptotic factor and ultimately blocks formation of a Bcl-2 heterodimer.

Many cancer drugs induce apoptosis in cancer cells by blocking formation of anti-apoptotic Bcl-2 heterodimers. The presence of a particular anti-apoptotic Bcl-2 heterodimer selected from in a cancer patient indicates that this patient is sensitive to a drug that interferes with formation of this anti-apoptotic Bcl-2 heterodimer. (See Robert et al., Clinical Pharmacology and Therapeutics 101;1, January 2017). On the other hand, apoptosis inhibitors can be used for treating neurodegenerative disease or cardiovascular disease, both of which involve apoptosis. In this context, the presence of a particular pro-apoptotic Bcl-2 heterodimer in a neurodegenerative disease patient or a cardiovascular disease patient, for example, indicates that such a patient is sensitive to an apoptosis inhibitor that blocks formation of the particular pro-apoptotic Bcl-2 heterodimer.

In some embodiments, the sensitivity is characterized by (a) the presence of apoptosis in the sample; (b) the presence of an anti-apoptotic Bcl-2 heterodimer selected from MCL-1 and BIM in the sample, indicating the patient is sensitive to a drug that interferes with formation of the heterodimer; (c) genetic risk factors; family history; personal history; race and ethnicity; features of the certain tissues; various benign conditions (e.g. nonproliferative lesions); previous chest radiation; carcinogen exposure and the like.

In some embodiments, the method does not involve a functional readout of mitochondrial outer membrane permeabilization (MOMP). In some embodiments, the method does not involve a dye-based detection of cell membrane potential.

### Illustrative Clinical Decisions

In some embodiments, the methods described herein are useful in the evaluation of a solid tumor or liquid tumor sample from a patient, or a blood cancer sample from a patient, for example, for evaluating diagnosis, prognosis, and response to treatment. In various aspects, the present disclosure comprises evaluating a solid tumor, liquid tumor, or a blood cancer sample from a patient. In various embodiments, the evaluation may be selected from diagnosis, prognosis, and response to treatment.

In various aspects, the methods of the present disclosure may be used to treat a cancer patient. For example, the methods may further comprise administering a cancer treatment to the patient if the ratio is predictive of sensitivity to the cancer treatment. In some embodiments, the methods may further comprise treating the patient with a reduced dose or less frequent and/or shortened regimen of the cancer treatment if the ratio is predictive of sensitivity to the cancer treatment. In some embodiments, the methods may further comprise treating the patient with an increased dose or more frequent and/or prolonged regimen of the cancer treatment if the ratio is predictive of sensitivity to the cancer treatment. In some embodiments, the methods may further comprise withholding cancer treatment to the patient if the ratio is predictive of a lack of sensitivity to the cancer treatment. In some embodiments, the methods may further comprise treating the patient with a different cancer treatment if the ratio is predictive of a lack of sensitivity to the cancer treatment.

For instance, in various embodiments, the sample presents a ratio of more dimer than monomer. For instance, the ratio of dimer to monomer may be about 20:1 or about 15:1, or about 10:1, or about 9:1, or about 8:1, or about 7:1, or about 6:1, or about 5:1, or about 4:1, or about 3:1, or about 2:1. In various embodiments, the sample presents a ratio of more monomer than dimer. For instance, the ratio of monomer to dimer may be about 20:1 or about 15:1, or about 10:1, or about 9:1, or about 8:1, or about 7:1, or about 6:1, or about 5:1, or about 4:1, or about 3:1, or about 2:1. In various embodiments, the ratio of dimer to monomer is equivalent (i.e, about 1:1).

Diagnosis refers to the process of attempting to determine or identify a possible disease or disorder, such as, for example, cancer. Prognosis refers to predicting a likely outcome of a disease or disorder, such as, for example, cancer. A complete prognosis often includes the expected duration, the function, and a description of the course of the disease, such as progressive decline, intermittent crisis, or sudden, unpredictable crisis. Response to treatment is a prediction of a patient's medical outcome when receiving a treatment. Responses to treatment can be, by way of non-limiting example, pathological complete response, survival, and progression free survival, time to progression, and probability of recurrence.

In various embodiments, the present methods direct a clinical decision regarding whether a patient is to receive a specific treatment. In one embodiment, the present methods are predictive of a positive response to neoadjuvant and/or adjuvant chemotherapy or a non-responsiveness to neoadjuvant and/or adjuvant chemotherapy. In one embodiment, the present methods are predictive of a positive response to a pro-apoptotic agent or an agent that operates via apoptosis and/or an agent that does not operate via apoptosis or a non-responsiveness to apoptotic effector agent and/or an agent that does not operate via apoptosis. In various embodiments, the present disclosure directs the treatment of a cancer patient, including, for example, what type of treatment should be administered or withheld.

In some embodiments, the present methods direct a cancer treatment regarding one or more of anti-cancer drugs, chemotherapy, antagonist of an anti-apoptotic protein, surgery, adjuvant therapy, and neoadjuvant therapy.

In one embodiment, the present methods direct a clinical decision regarding whether a patient is to receive adjuvant therapy after primary, main or initial treatment, including, without limitation, a single sole adjuvant therapy. Adjuvant therapy, also called adjuvant care, is treatment that is given in addition to the primary, main or initial treatment. By way of non-limiting example, adjuvant therapy may be an additional treatment usually given after surgery where all detectable disease has been removed, but where there remains a statistical risk of relapse due to occult disease.

In some embodiments, the present methods direct a patient's treatment to include adjuvant therapy. For example, a patient that is scored to be responsive to a specific treatment may receive such treatment as adjuvant therapy. Further, the present methods may direct the identity of an adjuvant therapy, by way of non-limiting example, as a treatment that induces and/or operates in a pro-apoptotic manner or one that does not. In one embodiment, the present methods may indicate that a patient will not be or will be less responsive to a specific treatment and therefore such a patient may not receive such treatment as adjuvant therapy. Accordingly, in some embodiments, the present methods provide for providing or withholding adjuvant therapy according to a patient's likely response. In this way, a patient's quality of life, and the cost of care, may be improved.

In various embodiments, the present methods direct a clinical decision regarding whether a patient is to receive neoadjuvant therapy, e.g. therapy to shrink and/or downgrade the tumor prior to surgery. In some embodiments, neoadjuvant therapy means chemotherapy administered to cancer patients prior to surgery. In some embodiments, neoadjuvant therapy means an agent, including those described herein, administered to cancer patients prior to surgery. Types of cancers for which neoadjuvant chemotherapy is commonly considered include, for example, breast, colorectal, ovarian, cervical, bladder, and lung.

In some embodiments, the present methods direct a patient's treatment to include neoadjuvant therapy. For example, a patient that is scored to be responsive to a specific treatment may receive such treatment as neoadjuvant therapy. Further, the present methods may direct the identity of a neoadjuvant therapy, by way of non-limiting example, as a treatment that induces and/or operates in a pro-apoptotic manner or one that does not. In one embodiment, the present methods may indicate that a patient will not be or will be less responsive to a specific treatment and therefore such a patient may not receive such treatment as neoadjuvant therapy. Accordingly, in some embodiments, the present methods provide for providing or withholding neoadjuvant therapy according to a patient's likely response. In this way, a patient's quality of life, and the cost of case, may be improved.

In some embodiments, the present methods direct a clinical decision regarding whether a patient is to receive a specific type of treatment (e.g., one or more of anti-cancer drugs, chemotherapy, antagonist of an anti-apoptotic protein, surgery, adjuvant therapy, and neoadjuvant therapy). In some embodiments, the cancer treatment is one or more of a SMAC mimetic, BH3 mimetic, proteasome inhibitor, histone deacetylase inhibitor, glucocorticoid, steroid, monoclonal antibody, antibody-drug conjugate, or thalidomide derivative. In some embodiments, the present methods are a guiding test for patient treatment.

In some embodiments, the present methods comprise a cancer treatment and the cancer treatment is a checkpoint inhibitor. The checkpoint inhibitor can be an agent that targets one of TIM-3, BTLA, PD-1, CTLA-4, B7-H4, GITR, galectin-9, HVEM, PD-L1, PD-L2, B7-H3,CD244, CD160, TIGIT, SIRPα, ICOS, CD172a, and TMIGD2. The agent that targets PD-1 can be an antibody or antibody format specific for PD-1, optionally selected from nivolumab, pembrolizumab, and pidilizumab. The agent that targets PD-L1 can be an antibody or antibody format specific for PD-L1, optionally selected from atezolizumab, avelumab, durvalumab, and BMS-936559. The agent that targets CTLA-4 can be an antibody or antibody format specific for CTLA-4, optionally selected from ipilimumab and tremelimumab.

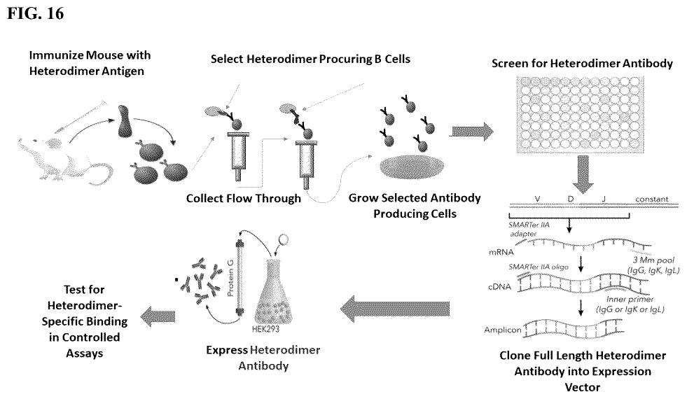

In some embodiments, the present methods comprise generating a MCL-1 and BIM heterodimer antibody as shown in FIG. 16. In some embodiments, the present methods comprise generating a MCL-1 and BIM heterodimer antibody comprising: (a) immunizing a subject with a heterodimer induced conformation antigen; (b) isolating from the subject a splenic B cell producing the IgG recognizing the heterodimer induced antigen; (c) passing the splenic B cell onto a magnetic column for negative selection, wherein the magnetic column for negative selection is coated with a recombinant fusion protein containing one monomer of the heterodimer; (d) collecting the flow through of the splenic B cells from the magnetic column for negative selection, and passing the flow through onto a magnetic column for positive selection; wherein the magnetic column for positive selection is coated with the heterodimer antigen; (e) eluting and collecting the splenic B cells bound to the magnetic column for positive selection; (f) culturing the collected cells in a B-cell media; and (g) isolating the heterodimer specific antibody from the cultured cells, thereby generating a heterodimer antibody. In some embodiments, the subject is a human, a monkey, a mouse, a rat, or a hamster.

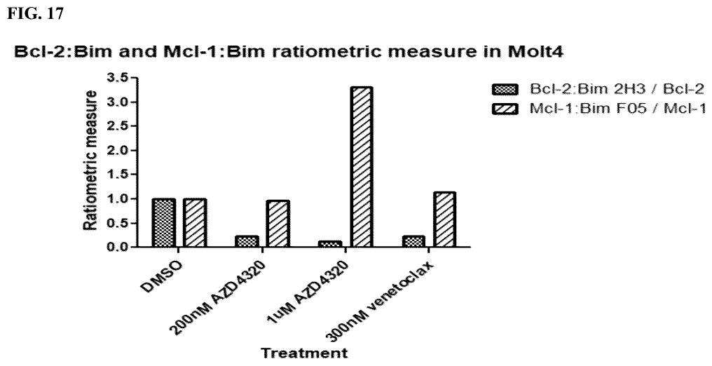

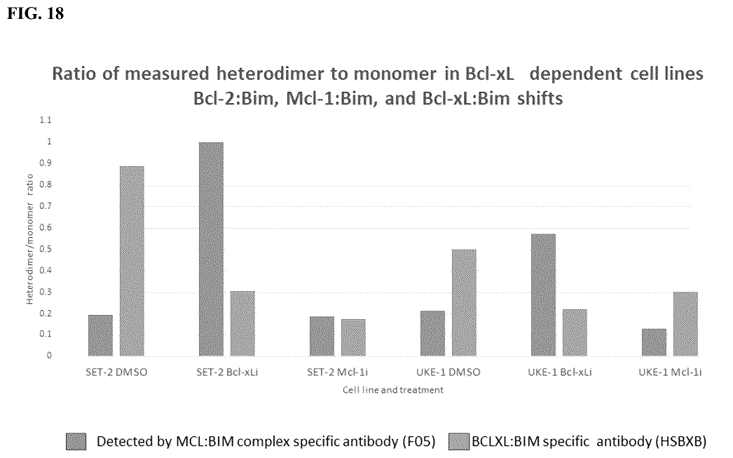

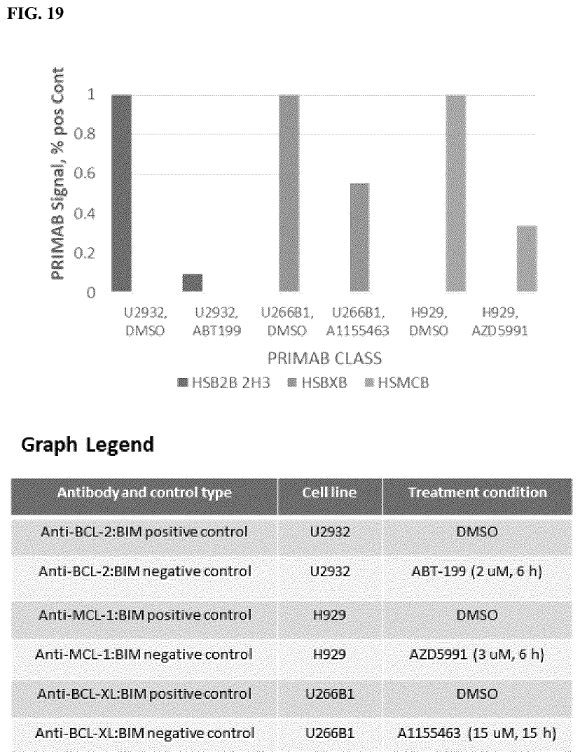

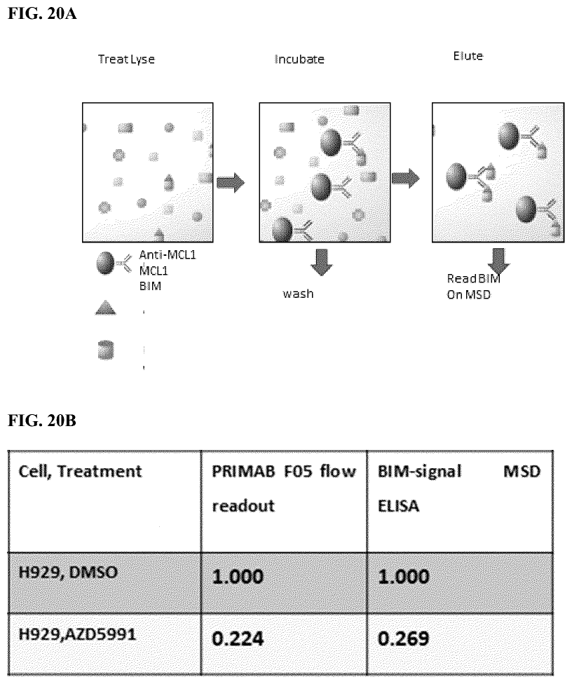

In some embodiments, the composition shifts a priming signal away from a BCL2:BIM complex, and towards a MCL-1:BIM complex (FIG. 17, FIG. 18, FIG. 19, FIG. 20A, FIG. 20B).

In some embodiments, the method futher comprises detecting a shift in a priming signal away from a BCL2:BIM complex, and towards a MCL-1:BIM complex (FIG. 17, FIG. 18, FIG. 19, FIG. 20A, FIG. 20B).

In some embodiments, the present methods provide information about the likely response that a patient is to have to a particular treatment. In some embodiments, the present methods provide a high likelihood of response and may direct treatment, including aggressive treatment. In some embodiments, the present methods provide a low likelihood of response and may direct cessation of treatment, including aggressive treatment, and the use of palliative care, to avoid unnecessary toxicity from ineffective chemotherapies for a better quality of life.

In an illustrative embodiment, the present method will indicate a likelihood of response to a specific treatment. For example, in some embodiments, the present methods indicate a high or low likelihood of response to a pro-apoptotic agent and/or an agent that operates via apoptosis and/or an agent that operates via apoptosis driven by direct protein modulation. In various embodiments, illustrative pro-apoptotic agents and/or agents that operate via apoptosis and/or an agent that operates via apoptosis driven by direct protein modulation include ABT-263 (Navitoclax), and obatoclax, WEP, bortezomib, Venetoclax (ABT-199), and carfilzomib. In some embodiments, the present methods indicate a high or low likelihood of response to an agent that does not operate via apoptosis and/or an agent that does not operate via apoptosis driven by direct protein modulation. In various embodiments, illustrative agents that do not operate via apoptosis include kinesin spindle protein inhibitors, cyclin-dependent kinase inhibitor, Arsenic Trioxide (TRISENOX), MEK inhibitors, pomolidomide, azacytidine, decitibine, vorinostat, entinostat, dinaciclib, gemtuzumab, BTK inhibitors, PI3 kinase delta inhibitors, lenolidimide, anthracyclines, cytarabine, melphalam, Aky inhibitors, mTOR inhibitors.

In an illustrative embodiment, the present method will indicate whether a patient is to receive a pro-apoptotic agent or an agent that operates via apoptosis for cancer treatment. In another illustrative embodiment, the present method will indicate whether a patient is to receive an agent that does not operate via apoptosis.

In a specific embodiment, the present methods are useful in predicting a cancer patient's response to any of the treatments (including agents) described herein.

In various embodiments, a cancer treatment is administered or withheld based on the methods described herein. Illustrative treatments include surgical resection, radiation therapy (including the use of the compounds as described herein as, or in combination with, radiosensitizing agents), chemotherapy, pharmacodynamic therapy, targeted therapy, immunotherapy, and supportive therapy (e.g., painkillers, diuretics, antidiuretics, antivirals, antibiotics, nutritional supplements, anemia therapeutics, blood clotting therapeutics, bone therapeutics, and psychiatric and psychological therapeutics).

### Illustrative Treatments

In illustrative embodiments, the disclosure selects a treatment agent. Examples of such agents include, but are not limited to, one or more of anti-cancer drugs, chemotherapy, surgery, adjuvant therapy, and neoadjuvant therapy. In one embodiment, the cancer treatment is one or more of a BH3 mimetic, epigenetic modifying agent, topoisomerase inhibitor, cyclin-dependent kinase inhibitor, and kinesin-spindle protein stabilizing agent. In some embodiments, the BH3 mimetic is selected from ABT-737 and ABT-263 (navitoclax), Bcl-2 specific Venetoclax (Venclexta, ABT-199), MCL-1 specific S63845 and AMG176 and ADZ5991, BCL-XL specific A-1155463 and A1331852, BFL-1/MCL-1 specific EU5346 or combinations thereof. In another embodiment, the cancer treatment is a proteasome inhibitor; and/or a modulator of cell cycle regulation (by way of non-limiting example, a cyclin dependent kinase inhibitor); and/or a modulator of cellular epigenetic mechanistic (by way of non-limiting example, one or more of a histone deacetylase (HDAC) (e.g. one or more of vorinostat or entinostat), azacytidine, decitabine); and/or an anthracycline or anthracenedione (by way of non-limiting example, one or more of epirubicin, doxorubicin, mitoxantrone, daunorubicin, idarubicin); and/or a platinum-based therapeutic (by way of non-limiting example, one or more of carboplatin, cisplatin, and oxaliplatin); cytarabine or a cytarabine-based chemotherapy; a BH3 mimetic (by way of non-limiting example, one or more of BCL2,BCLXL, or MCL1); an inhibitor of BIM, and an inhibitor of MCL-1. In some embodiments, the cancer treatment blocks formation of the particular heterodimer detected. In some embodiments, the cancer treatment perturbs or reduces formation of the particular heterodimer detected.

In various embodiments, the cancer treatment comprises one or more chemotherapeutic agents such as carboplatin, cisplatin, paclitaxel, gemcitabine, calicheamicin, doxorubicin, 5-fluorouracil, mitomycin C, actinomycin D, cyclophosphamide, vincristine, bleomycin, VEGF antagonists, EGFR antagonists, platins, taxols, irinotecan, 5-fluorouracil, gemcytabine, leucovorine, steroids, cyclophosphamide, melphalan, vinca alkaloids (e.g., vinblastine, vincristine, vindesine and vinorelbine), mustines, tyrosine kinase inhibitors, radiotherapy, sex hormone antagonists, selective androgen receptor modulators, selective estrogen receptor modulators, PDGF antagonists, TNF antagonists, IL-1 antagonists, interleukins (e.g. IL-12 or IL-2), IL-12R antagonists, Toxin conjugated monoclonal antibodies, Erbitux, Avastin, Pertuzumab, anti-CD20 antibodies, Rituxan, ocrelizumab, ofatumumab, DXL625, HERCEPTIN®, or any combination thereof.

In various embodiments, the disclosure pertains to cancer treatments including, without limitation, one or more of alkylating agents such as thiotepa and CYTOXAN cyclosphosphamide; alkyl sulfonates such as busulfan, improsulfan and piposulfan; aziridines such as benzodopa, carboquone, meturedopa, and uredopa; ethylenimines and methylamelamines including altretamine, triethylenemelamine, trietylenephosphoramide, triethiylenethiophosphoramide and trimethylolomelamine; acetogenins (e.g., bullatacin and bullatacinone); a camptothecin (including the synthetic analogue topotecan); bryostatin; cally statin; CC-1065 (including its adozelesin, carzelesin and bizelesin synthetic analogues); cryptophycins (e.g., cryptophycin 1 and cryptophycin 8); dolastatin; duocarmycin (including the synthetic analogues, KW-2189 and CB 1-TM1); eleutherobin; pancratistatin; a sarcodictyin; spongistatin; nitrogen mustards such as chlorambucil, chlornaphazine, cholophosphamide, estramustine, ifosfamide, mechlorethamine, mechlorethamine oxide hydrochloride, melphalan, novembichin, phenesterine, prednimustine, trofosfamide, uracil mustard; nitrosureas such as carmustine, chlorozotocin, fotemustine, lomustine, nimustine, and ranimnustine; antibiotics such as the enediyne antibiotics (e.g., calicheamicin, especially calicheamicin gammall and calicheamicin omegall (see, e.g., Agnew, Chem. Intl. Ed. Engl., 33:183-186 (1994)); dynemicin, including dynemicin A; bisphosphonates, such as clodronate; an esperamicin; as well as neocarzinostatin chromophore and related chromoprotein enediyne antiobiotic chromophores), aclacinomysins, actinomycin, authramycin, azaserine, bleomycins, cactinomycin, carabicin, caminomycin, carzinophilin, chromomycinis, dactinomycin, daunorubicin, detorubicin, 6-diazo-5-oxo-L-norleucine, ADRIAMYCIN doxorubicin (including morpholino-doxorubicin, cyanomorpholino-doxorubicin, 2-pyrrolino-doxorubicin and deoxy doxorubicin), epirubicin, esorubicin, idarubicin, marcellomycin, mitomycins such as mitomycin C, mycophenolic acid, nogalamycin, olivomycins, peplomycin, potfiromycin, puromycin, quelamycin, rodorubicin, streptonigrin, streptozocin, tubercidin, ubenimex, zinostatin, zorubicin; anti-metabolites such as methotrexate and 5-fluorouracil (5-FU); folic acid analogues such as denopterin, methotrexate, pteropterin, trimetrexate; purine analogs such as fludarabine, 6-mercaptopurine, thiamiprine, thioguanine; pyrimidine analogs such as ancitabine, azacitidine, 6-azauridine, carmofur, cytarabine, dideoxyuridine, doxifluridine, enocitabine, floxuridine; androgens such as calusterone, dromostanolone propionate, epitiostanol, mepitiostane, testolactone; anti-adrenals such as minoglutethimide, mitotane, trilostane; folic acid replenisher such as frolinic acid; aceglatone; aldophosphamide glycoside; aminolevulinic acid; eniluracil; amsacrine; bestrabucil; bisantrene; edatraxate; demecolcine; diaziquone; elformithine; elliptinium acetate; an epothilone; etoglucid; gallium nitrate; hydroxyurea; lentinan; lonidainine; maytansinoids such as maytansine and ansamitocins; mitoguazone; mitoxantrone; mopidanmol; nitraerine; pentostatin; phenamet; pirarubicin; losoxantrone; podophyllinic acid; 2-ethylhydrazide; procarbazine; PSK polysaccharide complex (JHS Natural Products, Eugene, Oreg.); razoxane; rhizoxin; sizofuran; spirogermanium; tenuazonic acid; triaziquone; 2,2′,2″-trichlorotriethylamine; trichothecenes (e.g., T-2 toxin, verracurin A, roridin A and anguidine); urethan; vindesine; dacarbazine; mannomustine; mitobronitol; mitolactol; pipobroman; gacytosine; arabinoside (“Ara-C”); cyclophosphamide; thiotepa; taxoids, e.g., TAXOL paclitaxel (Bristol-Myers Squibb Oncology, Princeton, N.J.), ABRAXANE Cremophor-free, albumin-engineered nanoparticle formulation of paclitaxel (American Pharmaceutical Partners, Schaumberg, 111.), and TAXOTERE doxetaxel (Rhone-Poulenc Rorer, Antony, France); chloranbucil; GEMZAR gemcitabine; 6-thioguanine; mercaptopurine; methotrexate; platinum analogs such as cisplatin, oxaliplatin and carboplatin; vinblastine; platinum; etoposide (VP-16); ifosfamide; mitoxantrone; vincristine; NAVELBINE, vinorelbine; novantrone; teniposide; edatrexate; daunomycin; aminopterin; xeloda; ibandronate; irinotecan (Camptosar, CPT-11) (including the treatment regimen of irinotecan with 5-FU and leucovorin); topoisomerase inhibitor RFS 2000; difluoromethylornithine (DMFO); retinoids such as retinoic acid; capecitabine; combretastatin; leucovorin (LV); oxaliplatin, including the oxaliplatin treatment regimen (FOLFOX); lapatinib (Tykerb); inhibitors of PKC-α, Raf, H-Ras, EGFR (e.g., erlotinib (Tarceva)) and VEGF-A that reduce cell proliferation, dacogen, velcade, and pharmaceutically acceptable salts, acids or derivatives of any of the above.

### Illustrative Detection Methods

In various embodiments, the present methods comprise predicting a patient's sensitivity to a cancer treatment. In some embodiments, the detection of the heterodimer employs an immunohistochemistry (IHC), flow cytometry, or immunofluorescent method.

In various embodiments, the methods involve evaluating a presence, absence, or level of a protein and/or a nucleic acid. In various embodiments, the present methods comprise evaluating a presence, absence, or level of a protein and/or a nucleic acid which can enhance the specificity and/or sensitivity of a MCL-1 and BIM heterodimer ratio. In some embodiments, the evaluating is of a marker for patient response. In some embodiments, the present methods comprise measurement using one or more of immunohistochemical staining (i.e., IHC), western blotting, in cell western, immunofluorescent staining, ELISA, and fluorescent activating cell sorting (FACS), or any other method described herein or known in the art. The present methods may comprise contacting an antibody with a tumor specimen (e.g. biopsy or tissue or body fluid) to identify an epitope that is specific to the tissue or body fluid and that is indicative of a state of a cancer.

There are generally two strategies used for detection of epitopes on antigens in body fluids or tissues, direct methods and indirect methods. The direct method comprises a one-step staining, and may involve a labeled antibody (e.g. FITC conjugated antiserum) reacting directly with the antigen in a body fluid or tissue sample. The indirect method comprises an unlabeled primary antibody that reacts with the body fluid or tissue antigen, and a labeled secondary antibody that reacts with the primary antibody. Labels can include radioactive labels, fluorescent labels, hapten labels such as, biotin, or an enzyme such as horse radish peroxidase or alkaline phosphatase. Methods of conducting these assays are well known in the art. See, e.g., Harlow et al. (Antibodies, Cold Spring Harbor Laboratory, NY, 1988), Harlow et al. (Using Antibodies, A Laboratory Manual, Cold Spring Harbor Laboratory, NY, 1999), Virella (Medical Immunology, 6th edition, Informa HealthCare, New York, 2007), and Diamandis et al. (Immunoassays, Academic Press, Inc., New York, 1996). Kits for conducting these assays are commercially available from, for example, Clontech Laboratories, LLC. (Mountain View, CA).

In various embodiments, antibodies include whole antibodies and/or any antigen binding fragment (e.g., an antigen-binding portion) and/or single chains of these (e.g. an antibody comprising at least two heavy (H) chains and two light (L) chains inter-connected by disulfide bonds, an Fab fragment, a monovalent fragment consisting of the V_{L}, V_{H}, C_{L} and CH1 domains; a F(ab)₂ fragment, a bivalent fragment including two Fab fragments linked by a disulfide bridge at the hinge region; a Fd fragment consisting of the V_{H} and CH1 domains; a Fv fragment consisting of the V_{L} and V_{H} domains of a single arm of an antibody; and the like). In various embodiments, polyclonal and monoclonal antibodies are useful, as are isolated human or humanized antibodies, or functional fragments thereof.

Standard assays to evaluate the binding ability of the antibodies toward the target of various species are known in the art, including for example, ELISAs, western blots and RIAs. The binding kinetics (e.g., binding affinity) of antibodies also can be assessed by standard assays known in the art, such as by Biacore analysis.

In another embodiment, the measurement comprises evaluating a presence, absence, or level of a nucleic acid. A person skilled in the art will appreciate that a number of methods can be used to detect or quantify the DNA/RNA levels of appropriate markers.

Gene expression can be measured using, for example, low-to-mid-plex techniques, including but not limited to reporter gene assays, Northern blot, fluorescent in situ hybridization (FISH), and reverse transcription PCR (RT-PCR). Gene expression can also be measured using, for example, higher-plex techniques, including but not limited, serial analysis of gene expression (SAGE), DNA microarrays. Tiling array, RNA-Seq/whole transcriptome shotgun sequencing (WTSS), high-throughput sequencing, multiplex PCR, multiplex ligation-dependent probe amplification (MLPA), DNA sequencing by ligation, and Luminex/XMAP. A person skilled in the art will appreciate that a number of methods can be used to detect or quantify the level of RNA products of the biomarkers within a sample, including arrays, such as microarrays, RT-PCR (including quantitative PCR), nuclease protection assays and Northern blot analyses.

### Illustrative Cancers and Patients

In some embodiments the disclosure provides a method for determining a cancer treatment and/or comprises a patient's tumor or cancer cell specimen. A cancer or tumor refers to an uncontrolled growth of cells and/or abnormal increased cell survival and/or inhibition of apoptosis which interferes with the normal functioning of the bodily organs and systems. A subject that has a cancer or a tumor is a subject having objectively measurable cancer cells present in the subject's body. Included in this disclosure are benign and malignant cancers, as well as dormant tumors or micrometastases. Cancers which migrate from their original location and seed vital organs can eventually lead to the death of the subject through the functional deterioration of the affected organs.

In various embodiments, the disclosure is applicable to pre-metastatic cancer, or metastatic cancer. Metastasis refers to the spread of cancer from its primary site to other places in the body. Cancer cells can break away from a primary tumor, penetrate into lymphatic and blood vessels, circulate through the bloodstream, and grow in a distant focus (metastasize) in normal tissues elsewhere in the body. Metastasis can be local or distant. Metastasis is a sequential process, contingent on tumor cells breaking off from the primary tumor, traveling through the bloodstream, and stopping at a distant site. At the new site, the cells establish a blood supply and can grow to form a life-threatening mass. Both stimulatory and inhibitory molecular pathways within the tumor cell regulate this behavior, and interactions between the tumor cell and host cells in the distant site are also significant. Metastases are often detected through the sole or combined use of magnetic resonance imaging (MRI) scans, computed tomography (CT) scans, blood and platelet counts, liver function studies, chest X-rays and bone scans in addition to the monitoring of specific symptoms.

The methods described herein are directed toward the prognosis of cancer, diagnosis of cancer, treatment of cancer, and/or the diagnosis, prognosis, treatment, prevention or amelioration of growth, progression, and/or metastases of malignancies and proliferative disorders associated with increased cell survival, or the inhibition of apoptosis. In some embodiments, the cancer is a solid tumor, including, but not limited to, non-small lung cell carcinoma, ovarian cancer, and melanoma.

In some embodiments, the sample is an infiltrating lymphocyte of the patient.

In some embodiments, the solid tumor is selected from lung cancer, breast cancer, prostate cancer, melanoma, pancreatic cancer, kidney cancer, colon cancer, and ovarian cancer. In some embodiments, the lung cancer is selected from non-small cell lung cancer (NSCLC) and small cell lung cancer (SCLC). In some embodiments, the breast cancer is triple negative breast cancer. In some embodiments, the prostate cancer is androgen independent prostate cancer.

In some embodiments, the sample is a liquid tumor, or a blood cancer. For example, hematopoietic cells are blood-forming cells in the body. Hematopoiesis (development of blood cells) begins in the bone marrow and depending on the cell type, further maturation occurs either in the periphery or in secondary lymphoid organs such as the spleen or lymph nodes. Hematopoietic disorders are recognized as clonal diseases, which are initiated by somatic and/or inherited mutations that cause dysregulated signaling in a progenitor cell. The wide range of possible mutations and accompanying signaling defects accounts for the diversity of disease phenotypes observed within this group of disorders. Hematopoietic disorders fall into three major categories: Myelodysplastic syndromes, myeloproliferative disorders, and acute leukemias. Examples of hematopoietic disorders include non-B lineage derived, such as acute myeloid leukemia (AML), Chronic Myeloid Leukemia (CML), non-B cell acute lymphocytic leukemia (ALL), myelodysplastic disorders, myeloproliferative disorders, polycythemias, thrombocythemias, or non-B atypical immune lymphoproliferations. Examples of B-Cell or B cell lineage derived disorder include Chronic Lymphocytic Leukemia (CLL), B lymphocyte lineage leukemia, Multiple Myeloma, acute lymphoblastic leukemia (ALL), B-cell pro-lymphocytic leukemia, precursor B lymphoblastic leukemia, hairy cell leukemia or plasma cell disorders, e.g., amyloidosis or Waldenstrom's macroglobulinemia.

Acute myeloid leukemia (AML), myelodysplastic syndrome (MDS), and myeloproliferative neoplasms (MPN) are examples of distinct myeloid hematopoietic disorders. However, it is recognized that these disorders share clinical overlap in that 30% of patients with MDS and 5-10% of patients with MPN will go on to develop AML. Below are current descriptions of these myeloid disorders.

AML is characterized by an uncontrolled proliferation of immature progenitor cells of myeloid origin including, but not limited to, myeloid progenitor cells, myelomonocytic progenitor cells, and immature megakaryoblasts. It is becoming clear that AML is really a heterogeneous collection of neoplasms with elements of differing pathophysiology, genetics and prognosis. Under WHO guidelines, diagnosis of AML can be made when blasts (immature cells) are present at 20% or more in peripheral blood or bone marrow sampling.

In some embodiments, the disclosure relates to one or more of the following cancers: adrenocortical carcinoma, AIDS-related cancers, anal cancer, appendix cancer, astrocytoma (e.g. childhood cerebellar or cerebral), basal-cell carcinoma, bile duct cancer, bladder cancer, bone tumor (e.g. osteosarcoma, malignant fibrous histiocytoma), brainstem glioma, brain cancer, brain tumors (e.g. cerebellar astrocytoma, cerebral astrocytoma/malignant glioma, ependymoma, medulloblastoma, supratentorial primitive neuroectodermal tumors, visual pathway and hypothalamic glioma), breast cancer, bronchial adenomas/carcinoids, carcinoid tumors, cerebellar astrocytoma, cervical cancer, chronic myeloproliferative disorders, colon cancer, desmoplastic small round cell tumor, endometrial cancer, ependymoma, esophageal cancer, Ewing's sarcoma, extracranial germ cell tumor, extragonadal germ cell tumor, extrahepatic bile duct cancer, eye cancer, gallbladder cancer, gastric (stomach) cancer, gastrointestinal stromal tumor (GIST), germ cell tumor (e.g. extracranial, extragonadal, ovarian), gestational trophoblastic tumor, gliomas (e.g. brain stem, cerebral astrocytoma, visual pathway and hypothalamic), gastric carcinoid, head and neck cancer, heart cancer, hepatocellular (liver) cancer, hypopharyngeal cancer, hypothalamic and visual pathway glioma, intraocular melanoma, islet cell carcinoma (endocrine pancreas), kidney cancer (renal cell cancer), laryngeal cancer, lip and oral cavity cancer, liposarcoma, liver cancer, lung cancer (e.g. non-small cell, small cell), medulloblastoma, melanoma, Merkel cell carcinoma, mesothelioma, metastatic squamous neck cancer, mouth cancer, multiple endocrine neoplasia syndrome, mycosis fungoides, myelodysplastic syndromes, myelodysplastic/myeloproliferative diseases, myeloproliferative disorders, chronic, nasal cavity and paranasal sinus cancer, nasopharyngeal carcinoma, neuroblastoma, non-small cell lung cancer, oral cancer, oropharyngeal cancer, osteosarcoma, ovarian cancer, pancreatic cancer, pancreatic cancer, paranasal sinus and nasal cavity cancer, parathyroid cancer, penile cancer, pharyngeal cancer, pheochromocytoma, pineal astrocytoma and/or germinoma, pineoblastoma and supratentorial primitive neuroectodermal tumors, pituitary adenoma, pleuropulmonary blastoma, prostate cancer, rectal cancer, renal cell carcinoma (kidney cancer), renal pelvis and ureter, retinoblastoma, rhabdomyosarcoma, salivary gland cancer, sarcoma (e.g. Ewing family, Kaposi, soft tissue, uterine), Sézary syndrome, skin cancer (e.g. nonmelanoma, melanoma, merkel cell), small cell lung cancer, small intestine cancer, soft tissue sarcoma, squamous cell carcinoma, squamous neck cancer, stomach cancer, supratentorial primitive neuroectodermal tumor, testicular cancer, throat cancerm, thymoma and thymic carcinoma, thyroid cancer, trophoblastic tumors, ureter and renal pelvis cancers, urethral cancer, uterine cancer, uterine sarcoma, vaginal cancer, visual pathway and hypothalamic glioma, vulvar cancer, Waldenström macroglobulinemia, and Wilms tumor.

The term subject, as used herein unless otherwise defined, is a mammal, e.g., a human, mouse, rat, hamster, guinea pig, dog, cat, horse, cow, goat, sheep, pig, or non-human primate, such as a monkey, chimpanzee, or baboon. The terms “subject” and “patient” are used interchangeably.

### Illustrative Specimens

In some embodiments, the present disclosure includes the measurement of a tumor specimen, including biopsy or surgical specimen samples. In some embodiments, the specimen is selected from a frozen tumor tissue specimen, cultured cells, circulating tumor cells, and a formalin-fixed paraffin-embedded tumor tissue specimen. In some embodiments, the biopsy is a human biopsy. In various embodiments, the biopsy is any one of a frozen tumor tissue specimen, cultured cells, circulating tumor cells, and a formalin-fixed paraffin-embedded tumor tissue specimen.

In some embodiments, the sample is selected from a tumor biopsy, tissue biopsy, tumor resection, frozen tumor tissue specimen, lymph node, bone marrow, circulating tumor cells, cultured cells, a formalin-fixed paraffin embedded tumor tissue specimen, bronchoalveolar lavage, skin, hair, urine, and combinations thereof. In some embodiments, the tumor biopsy is selected from a core biopsy, needle biopsy, surgical biopsy, and an excisional biopsy.

In some embodiments, the tumor specimen may be a biopsy sample, such as a frozen tumor tissue (cryosection) specimen. As is known in the art, a cryosection may employ a cryostat, which comprises a microtome inside a freezer. The surgical specimen is placed on a metal tissue disc which is then secured in a chuck and frozen rapidly to about −20° C, to about −30° C. The specimen is embedded in a gel like medium consisting of, for example, poly ethylene glycol and polyvinyl alcohol. The frozen tissue is cut frozen with the microtome portion of the cryostat, and the section is optionally picked up on a glass slide and stained.

In some embodiments, the tumor specimen may be a biopsy sample, such as cultured cells. These cells may be processed using the usual cell culture techniques that are known in the art. These cells may be circulating tumor cells.

In some embodiments, the tumor specimen may be a biopsy sample, such as a formalin-fixed paraffin-embedded (FFPE) tumor tissue specimen. As is known in the art, a biopsy specimen may be placed in a container with formalin (a mixture of water and formaldehyde) or some other fluid to preserve it. The tissue sample may be placed into a mold with hot paraffin wax. The wax cools to form a solid block that protects the tissue. This paraffin wax block with the embedded tissue is placed on a microtome, which cuts very thin slices of the tissue.

In certain embodiments, the tumor specimen (or biopsy) contains less than 100 mg of tissue, or in certain embodiments, contains about 50 mg of tissue or less. The tumor specimen (or biopsy) may contain from about 20 mg to about 50 mg of tissue, such as about 35 mg of tissue.

The tissue may be obtained, for example, as one or more (e.g., 1, 2, 3, 4, or 5) needle biopsies (e.g., using a 14-gauge needle or other suitable size). In some embodiments, the biopsy is a fine-needle aspiration in which a long, thin needle is inserted into a suspicious area and a syringe is used to draw out fluid and cells for analysis. In some embodiments, the biopsy is a core needle biopsy in which a large needle with a cutting tip is used during core needle biopsy to draw a column of tissue out of a suspicious area. In some embodiments, the biopsy is a vacuum-assisted biopsy in which a suction device increases the amount of fluid and cells that is extracted through the needle. In some embodiments, the biopsy is an image-guided biopsy in which a needle biopsy is combined with an imaging procedure, such as, for example, X ray, computerized tomography (CT), magnetic resonance imaging (MRI) or ultrasound. In other embodiments, the sample may be obtained via a device such as the MAMMOTOME® biopsy system, which is a laser guided, vacuum-assisted biopsy system for breast biopsy.

In certain embodiments, the specimen is a human tumor-derived cell line. In certain embodiments, the specimen is a cancer stem cell. In other embodiments, the specimen is derived from the biopsy of a solid tumor, such as, for example, a biopsy of a colorectal, breast, prostate, lung, pancreatic, renal, or ovarian primary tumor.

In certain embodiments, the specimen is of epithelial origin. In some embodiments, the epithelial specimen is enriched by selection from a biopsy sample with an anti-epithelial cell adhesion molecule (EpCAM) or other epithelial cell binding antibody bound to solid matrix or bead.

In certain embodiments, the specimen is of mesenchymal origin. In some embodiments, the mesenchymal specimen is enriched by selection from a biopsy sample with a neural cell adhesion molecule (N-CAM) or neuropilin or other mesenchymal cell binding antibody bound to a solid matrix or bead.

In some embodiments, the specimen is derived from a circulating tumor cell.

### Illustrative Clinical Factors and Additional Biomarkers

In some embodiments, the disclosure comprises determining one or more clinical factors of the patient. The disclosure can comprise detecting a heterodimer comprising two B-cell lymphoma 2 (BCL-2) proteins selected from MCL-1 and BIM in a solid tumor or liquid tumor sample from a patient, and determining a ratio of the heterodimer and/or clinical factors to assess a patient response or predict a patient's sensitivity to cancer treatment. In some embodiments, the clinical factor comprises further classifying the patient for likelihood of clinical response to the cancer treatment based on one or more clinical factors of the patient. In some embodiments, the clinical factor comprises comparing the prediction of the patient's sensitivity to the cancer treatment with the likelihood of clinical response to the cancer treatment based on one or more clinical factors of the patient. In some embodiments, a clinical factor that provides patient response information in combination with the ratio study may not be linked to apoptosis. In some embodiments, a clinical factor is non-apoptosis affecting.

In some embodiments, the clinical factor is one or more of age, cytogenetic status, performance, histological subclass, gender, and disease stage. In some embodiments, the clinical factor further comprises measuring an additional biomarker selected from mutational status, single nucleotide polymorphisms, steady state protein levels, and dynamic protein levels.

In one embodiment, the clinical factor is age. In one embodiment, the patient age profile is classified as over about 10, or over about 20, or over about 30, or over about 40, or over about 50, or over about 60, or over about 70, or over about 80 years old.

In one embodiment, the clinical factor is cytogenetic status. In some cancers, such as Wilms tumor and retinoblastoma, for example, gene deletion or inactivation are responsible for initiating cancer progression, as chromosomal regions associated with tumor suppressors are commonly deleted or mutated. For example, deletions, inversions, and translocations are commonly detected in chromosome region 9p21 in gliomas, non-small-cell lung cancers, leukemia's, and melanomas. Without wishing to be bound by theory, these chromosomal changes may inactivate the tumor suppressor cyclin-dependent kinase inhibitor 2A. Along with these deletions of specific genes, large portions of chromosomes can also be lost. For instance, chromosomes 1p and 16q are commonly lost in solid tumor cells. Gene duplications and increases in gene copy numbers can also contribute to cancer and can be detected with transcriptional analysis or copy number variation arrays. For example, the chromosomal region 12q13-q14 is amplified in many sarcomas. This chromosomal region encodes a binding protein called MDM2, which is known to bind to a tumor suppressor called p53. When MDM2 is amplified, it prevents p53 from regulating cell growth, which can result in tumor formation. Further, certain breast cancers are associated with overexpression and increases in copy number of the ERBB2 gene, which codes for human epidermal growth factor receptor 2. Also, gains in chromosomal number, such as chromosomes 1q and 3q, are also associated with increased cancer risk.

Cytogenetic status can be measured in a variety of manners known in the art. For example, FISH, traditional karyotyping, and virtual karyotyping (e.g. comparative genomic hybridization arrays, CGH and single nucleotide polymorphism arrays) may be used. For example, FISH may be used to assess chromosome rearrangement at specific loci and these phenomena are associated with disease risk status. In some embodiments, the cytogenetic status is favorable, intermediate, or unfavorable.

In one embodiment, the clinical factor is performance. Performance status can be quantified using any system and methods for scoring a patient's performance status are known in the art. The measure is often used to determine whether a patient can receive chemotherapy, adjustment of dose adjustment, and to determine intensity of palliative care. There are various scoring systems, including the Karnofsky score and the Zubrod score. Parallel scoring systems include the Global Assessment of Functioning (GAF) score, which has been incorporated as the fifth axis of the Diagnostic and Statistical Manual (DSM) of psychiatry. Higher performance status (e.g., at least 80%, or at least 70% using the Karnofsky scoring system) may indicate treatment to prevent progression of the disease state, and enhance the patient's ability to accept chemotherapy and/or radiation treatment. For example, in these embodiments, the patient is ambulatory and capable of self-care. In other embodiments, the evaluation is indicative of a patient with a low performance status (e.g., less than 50%, less than 30%, or less than 20% using the Karnofsky scoring system), so as to allow conventional radiotherapy and/or chemotherapy to be tolerated. In these embodiments, the patient is largely confined to bed or chair and is disabled even for self-care.

The Karnofsky score runs from 100 to 0, where 100 is “perfect” health and 0 is death. The score may be employed at intervals of 10, where: 100% is normal, no complaints, no signs of disease; 90% is capable of normal activity, few symptoms or signs of disease, 80% is normal activity with some difficulty, some symptoms or signs; 70% is caring for self, not capable of normal activity or work; 60% is requiring some help, can take care of most personal requirements; 50% requires help often, requires frequent medical care; 40% is disabled, requires special care and help; 30% is severely disabled, hospital admission indicated but no risk of death; 20% is very ill, urgently requiring admission, requires supportive measures or treatment; and 10% is moribund, rapidly progressive fatal disease processes.

The Zubrod scoring system for performance status includes: 0, fully active, able to carry on all pre-disease performance without restriction; 1, restricted in physically strenuous activity but ambulatory and able to carry out work of a light or sedentary nature, e.g., light house work, office work; 2, ambulatory and capable of all self-care but unable to carry out any work activities, up and about more than 50% of waking hours; 3, capable of only limited self-care, confined to bed or chair more than 50% of waking hours; 4, completely disabled, cannot carry on any self-care, totally confined to bed or chair; 5, dead.

In one embodiment, the clinical factor is histological subclass. In some embodiments, histological samples of tumors are graded according to Elston & Ellis, Histopathology, 1991, 19:403-10, the contents of which are hereby incorporated by reference in their entirety.

In one embodiment, the clinical factor is gender. In one embodiment, the gender is male. In another embodiment the gender is female.

In one embodiment, the clinical factor is disease stage. By way of non-limiting example, using the overall stage grouping, Stage I cancers are localized to one part of the body; Stage II cancers are locally advanced, as are Stage III cancers. Whether a cancer is designated as Stage II or Stage III can depend on the specific type of cancer. In one non-limiting example, Hodgkin's disease, Stage II indicates affected lymph nodes on only one side of the diaphragm, whereas Stage III indicates affected lymph nodes above and below the diaphragm. The specific criteria for Stages II and III therefore differ according to diagnosis. Stage IV cancers have often metastasized, or spread to other organs or throughout the body.

In another embodiment, the method further comprises a measurement of an additional biomarker selected from mutational status, single nucleotide polymorphisms, steady state protein levels, and dynamic protein levels. In another embodiment, the method further comprises predicting a clinical response in the patient. In another embodiment, the clinical response is about 1, about 2, about 3, or about 5-year progression/event-free survival.

A variety of clinical factors have been identified, such as age profile and performance status. A number of static measurements of diagnosis have also been utilized, such as cytogenetics and molecular events including, without limitation, mutations in the genes MLL, AML/ETO, Flt3-ITD, NPM1 (NPMc+), CEBPα, IDH1, IDH2, RUNX1, ras, and WT1 and in the epigenetic modifying genes TET2 and ASXL, as well as changes in the cell signaling protein profile.

In some embodiments, the preventive methods comprise administering a treatment to a patient that is likely to be afflicted by cancer as guided by the methods described herein. In some embodiments, a subject is likely to be afflicted by cancer if the subject is characterized by one or more of a high risk for a cancer, a genetic predisposition to a cancer (e.g. genetic risk factors), a previous episode of a cancer (e.g. new cancers and/or recurrence), a family history of a cancer, exposure to a cancer-inducing agent (e.g. an environmental agent), and pharmacogenomics information (the effect of genotype on the pharmacokinetic, pharmacodynamic or efficacy profile of a therapeutic).

In some embodiments, a subject is likely to be afflicted by cancer if the subject is characterized by a high risk for a cancer. In some embodiments, a subject is likely to be afflicted by cancer if the subject is characterized by a genetic predisposition to a cancer. In some embodiments, a genetic predisposition to a cancer is a genetic clinical factor, as is known in the art. Such clinical factors may include, by way of example, MLH1, MSH2, MSH6, PMS1, PMS2 for at least colon, uterine, small bowel, stomach, urinary tract cancers. In some embodiments, a subject is likely to be afflicted by cancer if the subject is characterized by a previous episode of a cancer. In some embodiments, the subject has been afflicted with 1, or 2, or 3, or 4, or 5, or 6, previous episodes of cancer. In some embodiments, a subject is likely to be afflicted by cancer if the subject is characterized by a family history of a cancer. In some embodiments, a parent and/or grandparent and/or sibling and/or aunt/uncle and/or great aunt/great uncle, and/or cousin has been or is afflicted with a cancer. In some embodiments, a subject is likely to be afflicted by cancer if the subject is characterized by exposure to a cancer-inducing agent (e.g. an environmental agent). For example, exposing skin to strong sunlight is a clinical factor for skin cancer. By way of example, smoking is a clinical factor for cancers of the lung, mouth, larynx, bladder, kidney, and several other organs.

Further, in some embodiments, the any one of the following clinical factors may be useful in the methods described herein: gender; genetic risk factors; family history; personal history; race and ethnicity; features of the certain tissues; various benign conditions (e.g. non-proliferative lesions); previous chest radiation; carcinogen exposure and the like.

Further still, in some embodiments, the any one of the following clinical factors may be useful in the methods described herein: one or more of a cell surface marker CD33, a cell surface marker CD34, a FLT3 mutation status, a p53 mutation status, a phosphorylation state of MEK-1 kinase, and phosphorylation of serine at position 70 of Bcl-2.

In some embodiments, the clinical factor is expression levels of the cytokines, including, without limitation, interleukin-6. In some embodiments, interleukin-6 levels will correlate with likelihood of response in MM patients, including a poor patient prognosis or a good patient prognosis.

In some embodiments, the likelihood of response is determined by assessing a percent priming. In certain embodiments, the priming is defined by the following equation:

$$\% Priming = [ 100 * ( \frac{negative control AUC - Peptid \text{?} AUC}{negative control AUC - Positive {Control}_{avg} AUC} ) ] Peptid \text{?} + [ 100 * ( \frac{negative control AUC - {Peptide}_{2} AUC}{negative control AUC - Positive {Control}_{avg} AUC} ) ] {Peptide}_{2} + … / ( n peptides )$$

$$\text{?} \text{indicates text missing or illegible when filed}$$

wherein:
the AUC (area under a curve) is a sum of fluorescence measurements established by homogenous time-resolved fluorescence (HTRF) or mean signal intensity from fluorescence activated cell sorting (FACS), wherein the signal intensity is a single time point measurement that occurs between about 5 min and about 300 min after the start of priming;
the negative control comprises a baseline negative control for either an area under a curve or a signal intensity;
the positive control comprises a baseline positive control for either an area under a curve or a signal intensity (e.g., any uncoupling agent); and
the Peptide is one or more BH3 domain peptides, wherein (n) is normalized with the average number of replicates of the negative and positive controls.

In some embodiments, in combination with the preceding equation, the one or more clinical factors are selected to increase specificity and/or sensitivity of the BH3 profile for association with clinical response.

In some embodiments, the likelihood of response is determined by assessing a percent priming. In certain embodiments, the priming is defined by the following equation:

$$\% Priming = [ 100 * ( \frac{DMSO AUC - Peptid \text{?} AUC}{DMSO AUC - {CCCP}_{avg} AUC} ) ] Peptid \text{} \text{?} + [ 100 * ( \frac{DMSO AUC - {Peptide}_{2} AUC}{DMSO AUC - {CCCP}_{avg} AUC} ) ] {Peptide}_{2} + … / ( n peptides )$$

$$\text{?} \text{indicates text missing or illegible when filed}$$

wherein:
the AUC (area under a curve) is a sum of fluorescence measurements established by homogenous time-resolved fluorescence (HTRF) or mean signal intensity from fluorescence activated cell sorting (FACS), wherein the signal intensity is a single time point measurement that occurs between about 5 min and about 300 min after the start of priming;
the DMSO (Dimethyl sulfoxide) comprises a baseline negative control for either an area under a curve or a signal intensity;
the CCCP (Carbonyl cyanide m-chlorophenyl hydrazone) is a chemical inhibitor of oxidative phosphorylation and comprises an effector of protein synthesis by serving as uncoupling agent of the proton gradient established during the normal activity of electron carriers in the electron transport chain in the mitochondria, and the CCCP comprises a baseline positive control; and
the Peptide is one or more BH3 domain peptides, wherein (n) is normalized with the average number of replicates of the DMSO and CCCP controls.

In some embodiments, in combination with the preceding equation, the one or more clinical factors are selected to increase specificity and/or sensitivity of the BH3 profile for association with clinical response.

In some embodiments, the likelihood of clinical response is defined by a simplified form of the preceding equation, as shown here:

$$\% Priming = [ 100 * ( \frac{{DMSO}_{avg} AUC - {Peptide}_{n} AUC}{{DMSO}_{avg} AUC - {CCCP}_{avg} AUC} ) ]$$

wherein:
the AUC (area under a curve) is a sum of fluorescence measurements established by homogenous time-resolved fluorescence (HTRF) or mean signal intensity from fluorescence activated cell sorting (FACS), wherein the signal intensity is a single time point measurement that occurs between about 5 min and about 300 min after the start of priming;
the DMSO (Dimethyl sulfoxide) comprises a baseline negative control for either an area under a curve or a signal intensity;
the CCCP (Carbonyl cyanide m-chlorophenyl hydrazone) is a chemical inhibitor of oxidative phosphorylation and comprises an effector of protein synthesis by serving as uncoupling agent of the proton gradient established during the normal activity of electron carriers in the electron transport chain in the mitochondria, and the CCCP comprises a baseline positive control; and
the Peptide is one or more BH3 domain peptides, wherein (n) is normalized with the average number of replicates of the DMSO and CCCP controls.

In some embodiments, in combination with the preceding equation, the one or more clinical factors are selected to increase specificity and/or sensitivity of the BH3 profile for association with clinical response.

In some embodiments, the area under the curve is established by homogenous time-resolved fluorescence (HTRF). In some embodiments, the time occurs over a window from between about 0 to about 300 min to about 0 to about 30 min. In some embodiments, the area under the curve is established by fluorescence activated cell sorting (FACS). In some embodiments, the signal intensity is a single time point measurement that occurs between about 5 min and about 300 min.

In some embodiments, the present disclosure provides a method for predicting a patient's responsiveness to a checkpoint inhibitor in a sample, comprising measuring the amount of a MCL-1/BIM heterodimer, wherein the sample comprises an infiltrating lymphocyte population from a solid tumor or liquid tumor. The checkpoint inhibitor can an agent that targets one of TIM-3, BTLA, PD-1, CTLA-4, B7-H4, GITR, galectin-9, HVEM, PD-L1, PD-L2, B7-H3, CD244, CD160, TIGIT, SIRPα, ICOS, CD172a, and TMIGD2. The agent that targets PD-1 can be an antibody or antibody format specific for PD-1, optionally selected from nivolumab, pembrolizumab, and pidilizumab. The agent that targets PD-L1 can an antibody or antibody format specific for PD-L1, optionally selected from atezolizumab, avelumab, durvalumab, and BMS-936559. The agent that targets CTLA-4 can be an antibody or antibody format specific for CTLA-4, optionally selected from ipilimumab and tremelimumab.

In some embodiments, the present methods comprise generating a MCL-1 and BIM heterodimer antibody as shown in FIG. 16. In some embodiments, the present methods comprise generating a MCL-1 and BIM heterodimer antibody comprising: (a) immunizing a subject with a heterodimer induced conformation antigen; (b) isolating from the subject a splenic B cell producing the IgG recognizing the heterodimer induced antigen; (c) passing the splenic B cell onto a magnetic column for negative selection, wherein the magnetic column for negative selection is coated with a recombinant fusion protein containing one monomer of the heterodimer; (d) collecting the flow through of the splenic B cells from the magnetic column for negative selection, and passing the flow through onto a magnetic column for positive selection; wherein the magnetic column for positive selection is coated with the heterodimer antigen; (e) eluting and collecting the splenic B cells bound to the magnetic column for positive selection; (f) culturing the collected cells in a B-cell media; and (g) isolating the heterodimer specific antibody from the cultured cells, thereby generating a heterodimer antibody. In some embodiments, the subject is a human, a monkey, a mouse, a rat, or a hamster.

In some embodiments, the present disclosure provides a polynucleotide comprising a nucleic acid sequence encoding the antibody or antibody fragment. In some embodiments, a vector comprising the polynucleotide provided; in some embodiments, a host cell comprising the vector is provided.

In some aspects, the present disclosure provides a pharmaceutical composition comprising the antibody or antibody format of any of the antibodies disclosed herein and a pharmaceutically acceptable excipient.

The disclosure also provides kits that can simplify the evaluation of tumor or cancer cell specimens. A typical kit of the disclosure comprises various reagents including, for example, one or more agents (e.g., an antibody as disclosed herein) useful to detect a heterodimer. The kit can further comprise materials necessary for the evaluation, including welled plates, syringes, and the like. The kit can further comprise a label or printed instructions instructing the use of described reagents. The kit can further comprise a treatment to be tested.

It should be understood that singular forms such as “a,” “an,” and “the” are used throughout this application for convenience, however, except where context or an explicit statement indicates otherwise, the singular forms are intended to include the plural. Further, it should be understood that every journal article, patent, patent application, publication, and the like that is mentioned herein is hereby incorporated by reference in its entirety and for all purposes. All numerical ranges should be understood to include each and every numerical point within the numerical range, and should be interpreted as reciting each and every numerical point individually. The endpoints of all ranges directed to the same component or property are inclusive, and intended to be independently combinable.

“About” includes all values having substantially the same effect, or providing substantially the same result, as the reference value. Thus, the range encompassed by the term “about” will vary depending on context in which the term is used, for instance the parameter that the reference value is associated with. Thus, depending on context, “about” can mean, for example, ±15%, ±10%, ±5%, ±4%, ±3%, ±2%, ±1%, or ±less than 1%. Importantly, all recitations of a reference value preceded by the term “about” are intended to also be a recitation of the reference value alone. Notwithstanding the preceding, in this application the term “about” has a special meaning with regard to pharmacokinetic parameters, such as area under the curve (including AUC, AUCₜ, and AUC_{∞}) Cₘₐₓ, Tₘₐₓ, and the like. When used in relationship to a value for a pharmacokinetic parameter, the term “about” means from 85% to 115% of the reference parameter.

As used herein, the word “include,” and its variants, is intended to be non-limiting, such that recitation of items in a list is not to the exclusion of other like items that may also be useful in the materials, compositions, devices, and methods of this technology. Similarly, the terms “can” and “may” and their variants are intended to be non-limiting, such that recitation that an embodiment can or may comprise certain elements or features does not exclude other embodiments of the present technology that do not contain those elements or features.

Although the open-ended term “comprising,” as a synonym of terms such as including. containing, or having, is used herein to describe and claim the disclosure, the present technology, or embodiments thereof, may alternatively be described using more limiting terms such as “consisting of” or “consisting essentially of” the recited ingredients.

Unless defined otherwise, all technical and scientific terms herein have the same meaning as commonly understood by one of ordinary skill in the art to which this disclosure belongs. Although any methods and materials, similar or equivalent to those described herein, can be used in the practice or testing of the present disclosure, the preferred methods and materials are described herein. All publications, patents, and patent publications cited are incorporated by reference herein in their entirety for all purposes.

This disclosure is further illustrated by the following non-limiting examples.

### EXAMPLES

### Example 1: Preparation of Monoclonal Antibodies Specific to Bcl-2 Heterodimers

Genes encoding human Mcl-1 were cloned and mutated to delete their transmembrane domains. The mutated genes were then linked to a nucleotide sequence encoding Glutathione-S-transferase (GST) and cloned into pGEX 4T-1 to obtain DNA constructs for expressing Mcl-1(Δ)-GST fusion proteins. DNA constructs for expressing full length human Bim, all fused with GST, were prepared by recombinant technology.

All of the DNA constructs were introduced into BL21 (D3) chemically competent E. coli cells. Positive transformants were cultured in a suitable medium and expression of the fusion proteins were induced with isopropyl-1-thio-β-D-galactopyranoside. The expressed fusion proteins were purified using Amersham Hitrap Glutathion e column on the ACTA-FPLC (Amersham) and accurately quantified using spectrophotometry.

Mcl-1(Δ)-GST wasthen mixed with Bim-GST at equamolar amounts in PBS.

Aromatic amino acids within the Bim BH3 peptides were derivatized with BPA as described in Table 2. Each of these peptides were tested for binding affinities using fluorescence polarization as Richard, D. J.; et al. Bioorg. Med. Chem. 2013 Nov. 1;21(21):6642-9.

TABLE 2
Bim BH3 domain containing peptides used to make
heterodimer

| Sequences: | U = bpa; Bpa = 4-benzoylphenylalanine |
|---|---|
| 1 | IUIAQELRRIGDEFNAYYARR |
| 2 | IWIAQELRRIGDEFNAUYARR |
| 3 | IWIAQELRRIGDEFNAYUARR |
| 4 | IWIAQELRRIGDEUNAYYARR |

Selected peptides were then coupled to purified GST-anti-apoptotic Mcl-1 fusion proteins. For this molar equivalents of each peptide and the MCL-1 GST fusion protein were combined in separate silconnized 300 ul reaction tubes and agitated. After a brief spin, samples were irradiated with UV light, (450 nM), by exposure to a 100 W halogen lamp for 30 minutes at room temperature. The protein peptide complexes were purified using a sepharose 12 column (Pharmacia) on a ACTA-FPLC (Amersham), following the method described in Zue et al., Protein Science 6:781-788 (2007). Successful coupling was determined using Matrix Assisted Laser Desorption/Ionization (Time of Flight (MALDI-TOF)) mass spectrometry. Reaction mixtures were coupled to reverse phase MALDI chips and analyzed using the Voyager (Applied Biosystems) mass spectrometer. Appropriate mass shifts were observed in the successful reaction samples. Samples were further assessed for coupling by using competitive FP as described in Richard, D. J. et al Bioorg. Med. Chem. 2013 Nov. 1; 21(21):6642-9. Complexes that were unable to bind FITC labeled BIM-BH3 peptides were excluded. Peptide sequence 4 was selected for the immunogen.

The heterodimer complex consisting of MCL-1-BIM BIM-BPA #4 from the table above (2.mg) was then suspended in monophosphoryl lipid A plus trehalose dicorynomycolate adjuvant (Ribi Immunochem. Research Inc., Hamilton, Mont.). The formed mixture was then injected into Balb/c mice at each hind foot pad once every 3-4 days for 14 times. Three days after the final injection, spleen cells were removed from the mice and a single cell suspension is prepared in a DMEM medium (Gibco/BRL Corp.) supplemented with 1% penicillin-streptomycin. The spleen cells were fused with murine myeloma cells P3X63AgU.1 (ATCC CRL 1597) using 35% polyethylene glycol and cultured in 96-well culture plates.

Hybridomas were selected in super DMEM (DMEM supplemented with 10% fetal calf serum FCS, 100 mM pyruvate, 100 U/ml insulin, 100 mM oxaloacetic acid, 2 mM glutamine, 1% nonessential amino acids (GIBCO/BRL), 100 U/ml penicillin, and 100 μg/ml streptomycin] containing 100 μM hypoxanthine, 0.4 μM aminopterin, and 16 μM thymidine (HAT), (Sigma Chemical Co., St. Louis, Mo.).

Hybridoma cells were fed with 200 μl of super DMEM containing 10% FCS and antibiotics. Ten days after the fusion, supernatants of the hybridoma cultures were collected and screened for the presence of antibodies that were bound to the cognate heterodimer protein and/or to either member of the heterodimer (as negative controls) in a capture ELISA as described in Certo et al., Cancer Cell., 9(5):351-365(2006).

Briefly, 96-well microtiter plates (Maxisorb; Nunc, Kamstrup, Denmark) were coated with 50 μl (1 μg/ml) of a heterodimer or a member of the heterodimer at 4° C. overnight. The plates were then washed three times with PBS containing 0.05% TWEEN 20™ (PBST) and blocked with 50 μl PBS containing 2.0% bovine serum albumin (BSA) at room temperature for 1 hour. The plates were then washed again three times with PBST. Afterwards, 100 μl of a hybridoma supernatant was added to designated wells. The plates were incubated at room temperature for 1 hour on a shaker apparatus and then washed three times with wash buffer. Next, 50 μl of HRP-conjugated goat anti-mouse IgG Fc (Cappel Laboratories), diluted 1:1000 in assay buffer (0.5% bovine serum albumin, 0.05% % TWEEN 20™, 0.01% Thimersol in PBS), was added to each well. The plates were then incubated for 1 hour at room temperature on a shaker apparatus and washed three times with wash buffer, followed by addition of 50 μl of substrate DACO and incubation at room temperature for 10 minutes. 50 μl of diethyl glycol was added to each well to stop the reaction and absorbance at 450 nm in each well is read in a microliter plate reader.

Hybridoma cells producing antibodies that bind to a MCL-1 and BIM heterodimer, but not to either member of the heterodimer, were then selected. These positive hybridoma cells were cloned twice and the specificity of the produced antibodies were retested. The isotypes of the antibodies having the desired specificity were determined by conventional methods, e.g., using isotype specific goat anti-mouse IgGs (Fisher Biotech, Pittsburgh, Pa.). The specificity of the antibodies in each antiserum was examined by conventional methods, e.g., the immunoprecipitation and FACS assays described in Examples 2-7 below.

### Example 2: Selective Binding of MCL-1 and BIM Heterodimers

Immunoassays (e.g., ELISA, immunoprecipitation assay) were performed to confirm that the antibodies were specific to a MCL-1 and BIM heterodimer. (FIG. 1 and FIG. 2) In the experiment shown in FIG. 1, Mcl-1-GST was bound to glutathione-coated ELISA plates. Bim-BH3 peptide was then added, or not added as a control, and HSMCB antibody was used to detect complex formation. Two HSMCB antibodies were tested: an antibody having CDRs selected from SEQ ID NO: 9, SEQ ID NO: 10, SEQ ID NO: 11, SEQ ID NO: 12, SEQ ID NO: 13, SEQ ID NO: 14, and having a variable heavy chain selected from SEQ ID NO: 15, and a variable light chain selected from SEQ ID NO: 16 (left panel), and an antibody having CDRs selected from SEQ ID NO: 1, SEQ ID NO: 2, SEQ ID NO: 3, SEQ ID NO: 4, SEQ ID NO: 5, SEQ ID NO: 6, and having a variable heavy chain selected from SEQ ID NO: 7, and a variable light chain selected from SEQ ID NO: 8 (right panel). The results of this experiment indicate highly selective binding of Mcl-1 and Bim peptide to the HSMCB heterodimer antibody, which demonstrates a dynamic range of binding. The results of this experiment indicate also that both HSMCB antibodies preferentially react with the heterodimer and not to the monomer.

### Example 3: Stability of HSMCB Antibodies

The experiments of this example demonstrate the selective binding of the Mcl-1/Bim heterodimer (HSMCB) antibody to the Mcl-1/Bim heterodimer for an antibody having CDRs selected from SEQ ID NO: 9, SEQ ID NO: 10, SEQ ID NO: 11, SEQ ID NO: 12, SEQ ID NO: 13, SEQ ID NO: 14, and having a variable heavy chain selected from SEQ ID NO: 15, and a variable light chain selected from SEQ ID NO: 16, and the stability of this antibody at 4° C. for about 2 months (FIG. 2). Each line in the graph follows the order in the legend (i.e., the top line is for sample 5-01-20b, the next line is for 5-22-20b, the next line is for 5-29b; this pattern is throughout the entire graph).

### Example 4: Immunofluorescence (IF) Stainings of the HSMCB Antibodies, and si-RNA Knockdown of Bim in Human Breast Cancer Cells

The experiments of this example show a set of IHC images relating to the application of the Mcl-1/Bim heterodimer antibody (HSMCB) to formalin-fixed paraffin-embedded (FFPE) specimens from MCF-7 breast cancer cells, and HCC70 triple negative breast cancer cells. (FIG. 3 and FIG. 4) The results of the experiment demonstrate that the Mcl-1/Bim heterodimer antibody (HSMCB) requires Bim to bind in situ. BIM siRNA was used on both MCF-7 and HCC70 cells (far left, and right middle columns, respectively), and the cells were then formalin-fixed and paraffin embedded into a tissue microarray (TMA) block, sectioned and added to glass slides (FIG. 3), and the cells were then stained with HSMCB. IHC images of the wild-type (WT) staining for both MCF-7 and HCC70 cells are shown in the middle-left, and far right columns, respectively. The IHC images show a reduced HSMCB IHC signal intensity in Bim-siRNA transfected MCF-7 cells, and Bim-siRNA transfected HCC70 cells.

In FIG. 4, IHC images are shown comparing the application of HSMCB, and the Bcl-xL/Bim heterodimer antibody (HSBXB), to formalin-fixed paraffin-embedded (FFPE) specimens from HCC70 cells. In the far left panel, IHC images of the HSMCB antibody are shown for both WT HCC70 cells, and Bim-siRNA transfected HCC70 cells. In the middle panel, IHC images of the HSBXB antibody are shown for both WT HCC70 cells, and Bim-siRNA transfected HCC70 cells. In the far right panel, IHC images are shown of HCC70 cells without application of HSMCB or HSBXB. The IHC images show reduced activity for both HSMCB and HSBXB in Bim-siRNA transfected HCC70 cells.

### Example 5: Quantitative Immunohistochemistry on PDX and Solid Tissue Samples

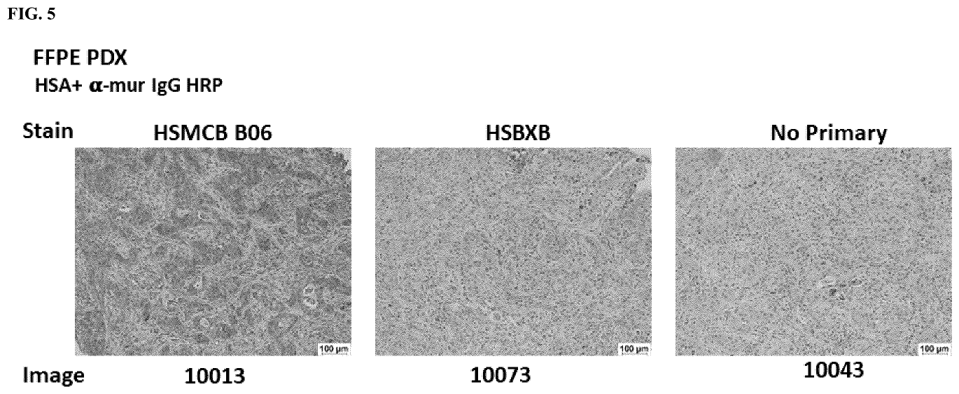

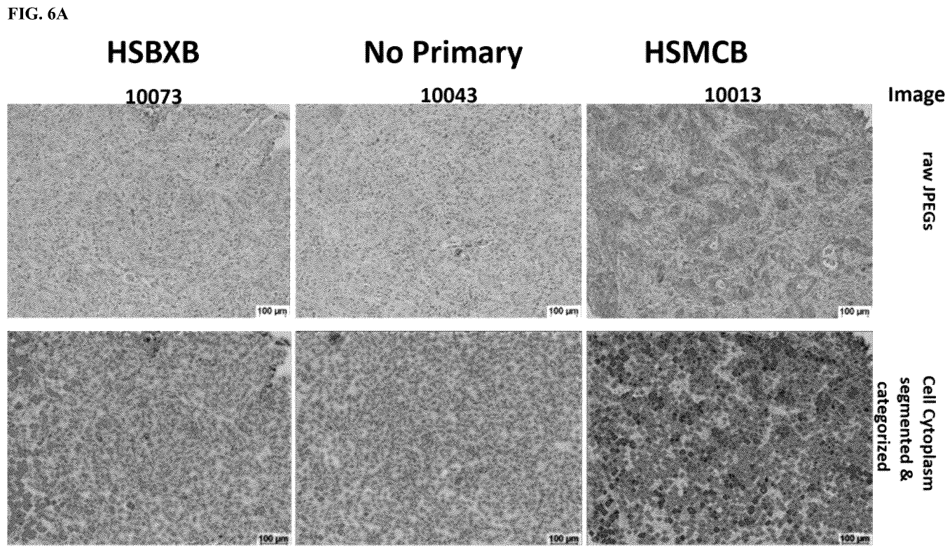

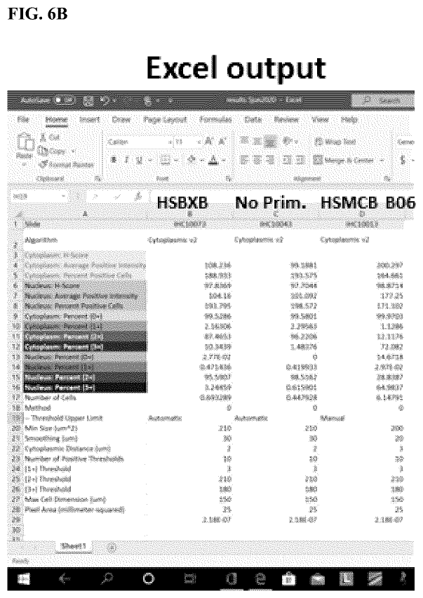

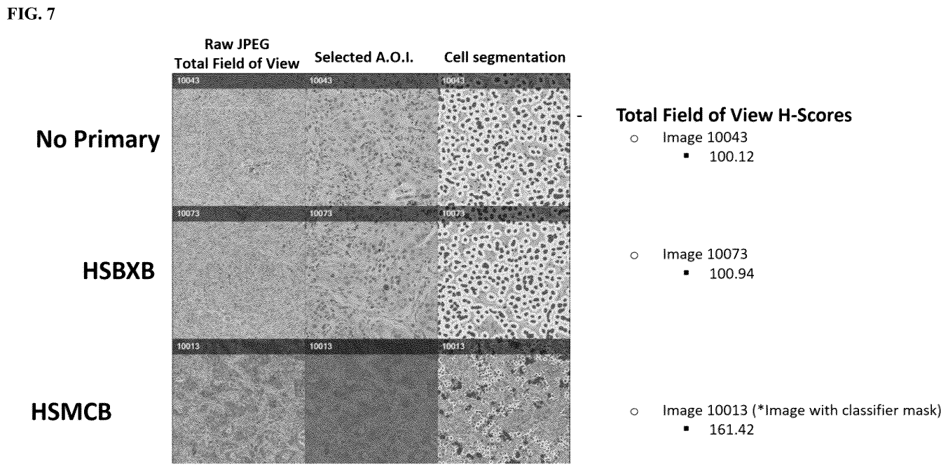

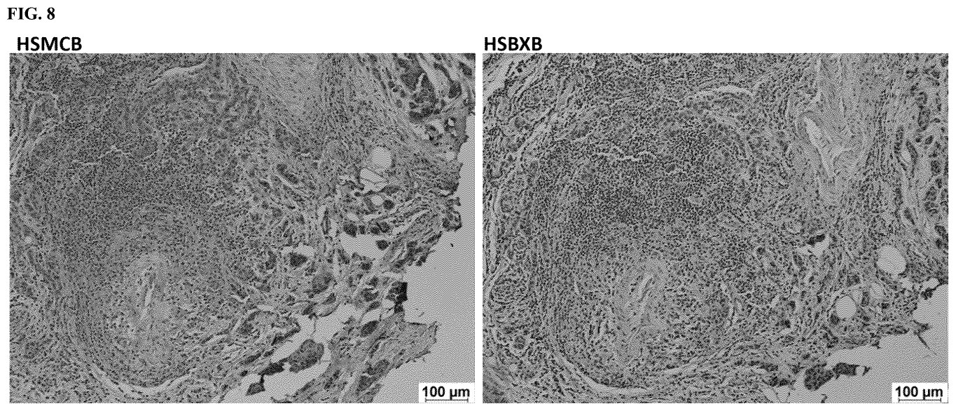

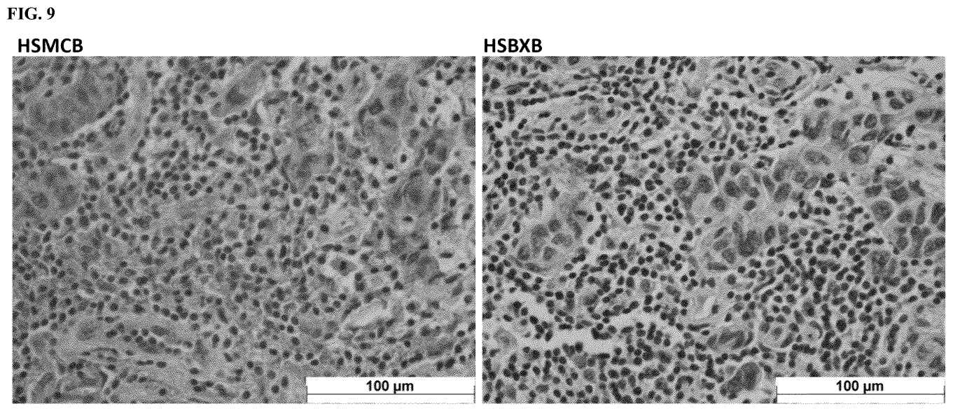

The experiments of this example demonstrate HSMCB and HSBXB staining using Leica or HALO image analysis software for quantitative analysis. In these experiments, PDX stains were used for image analysis software from Leica and Halo were compared for quantitative analysis. FIG. 5 shows IHC images comparing HSMCB and HSBXB reactivity with separate serial sections from the same FFPE patient derived xenotransplant (PDX) to test the performance of Leica and HALO image analysis software for performing segmentation of cancerous cells and the antibody signals within them. FIG. 6A shows IHC images of PDX Cell Segmentation Performance following Leica image analysis software applied cellular segmentation according to magnitude of cytoplasmic staining, and FIG. 6B shows quantitative analysis of the images. FIG. 7 shows IHC images of PDX Cell Segmentation Performance (by enlarged nucleus) using HALO image analysis software, and FIG. 8 shows IHC images of HSMCB (left column), and HSBXB staining (right column) with serial sections from a FFPE breast cancer patient biopsy (BCa_01 NV401) using a 10× objective. The HSMCB demonstrates higher reactivity with this tissue sample than HSBXB. FIG. 9 shows images from the same samples used for FIG. 8 acquired at a heightened magnification (40× objective) highlighting areas containing enlarged (and positively stained) cancerous cells amongst smaller tumor infiltrating lymphocytes.

### Example 6: Mcl-1/Bim (HSMCB) Heterodimer Signal Readout Guides Biomarker Development

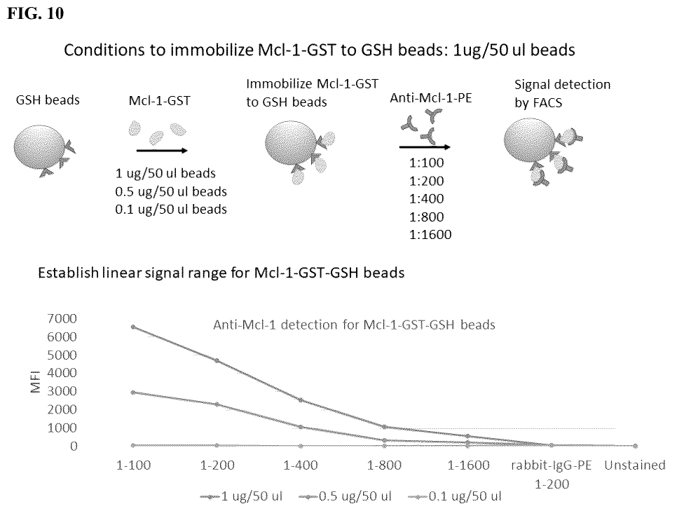

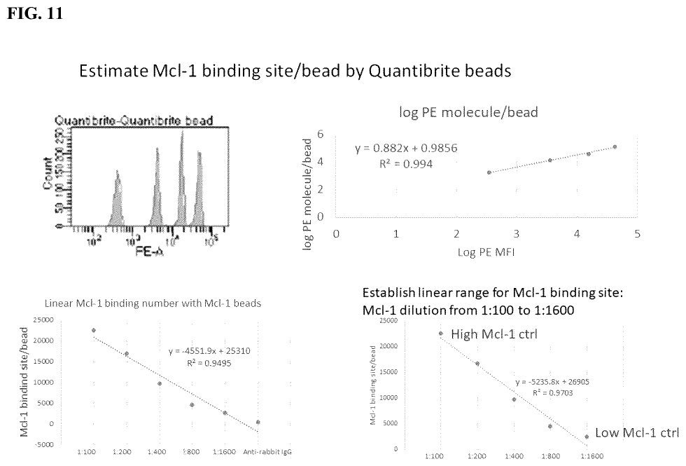

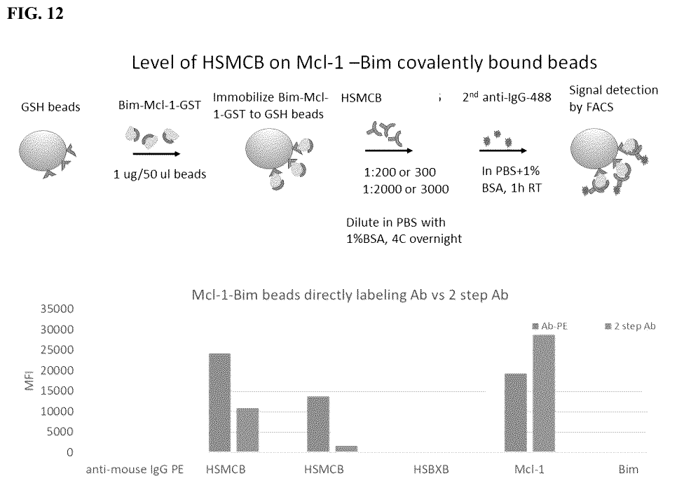

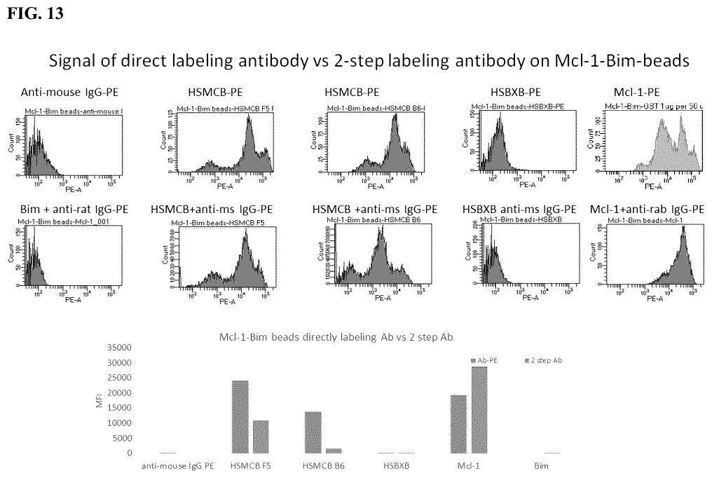

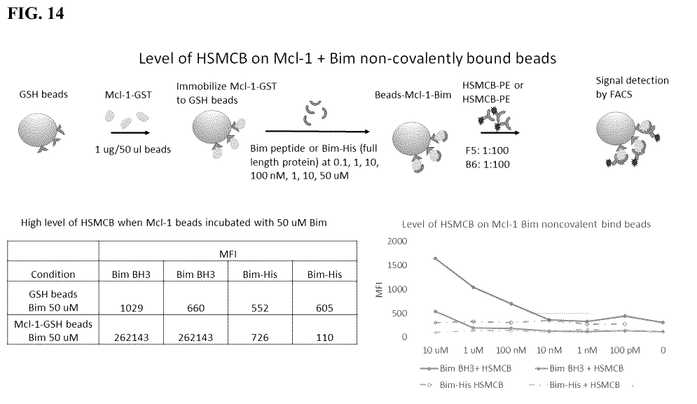

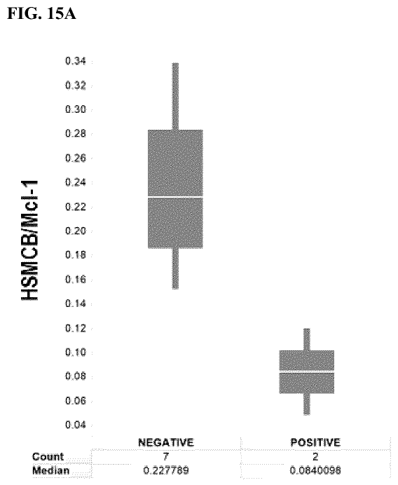

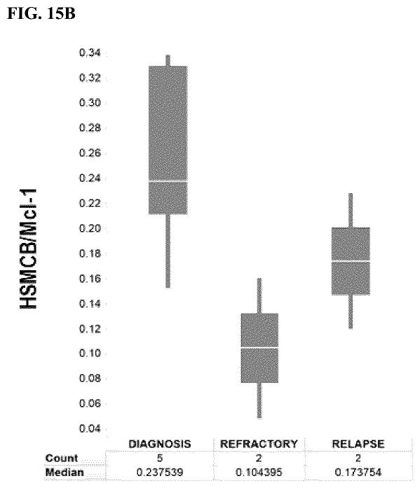

The experiments of this example show how the heterodimer-specific Mcl-1/Bim (HSMCB) monoclonal antibodies has been raised and engineered against covalent immunogens, and are being developed as clinical biomarkers for liquid and solid tumor therapies. The experiments of this example show that HSMCB specificity to Mcl-1/Bim has a dynamic range of signal, which has been established using ELISA, flow cytometry, and immunofluorescence in untreated and knockdown cell lines and in AML patient biopsies. (FIG. 10, FIG. 11, FIG. 12, FIG. 13, FIG. 14, FIG. 15A, and FIG. 15B) These HSMCB antibodies have utility in monitoring the shifts in the heterodimer complexes in conditions where Mcl-1 stability and complex formation is modulated or perturbed, for instance, by CDK9 inhibitor treatment. This feature indicates promise for use as a biomarker that can identify Mcl-1 survival dependencies and Bcl-2 inhibitor resistance in liquid and solid tumors.

AML patient samples were BH3 profiled in the experiments of this example. FIG. 10 shows the conditions to immobilize Mcl-1-GST to GSH beads, and detection for Mcl-1-GST-GSH beads using an anti-Mcl-1 antibody. FIG. 11 shows graphs that estimate the Mcl-1 binding site per bead by Quantibrite beads. FIG. 12 also shows detection of Mcl-1-Bim beads (bottom), and FIG. 13 shows a series of flow cytometry graphs each depicting the signal detected by direct labeling of the HSMCB antibody, compared to the signal obtained by two-step labeling of the HSMCB antibody on Mcl-Bim coated beads. FIG. 14 shows conditions to immobilize HSMCB on Mcl-1-Bim non-covalently bound beads. In the experiments of FIG. 14, an HSMCB antibody having CDRs selected from SEQ ID NO: 1, SEQ ID NO: 2, SEQ ID NO: 3, SEQ ID NO: 4, SEQ ID NO: 5, SEQ ID NO: 6, and having a variable heavy chain selected from SEQ ID NO: 7, and a variable light chain selected from SEQ ID NO: 8 was shown to recognize the Mcl-1 Bim-BH3 complex at a significant level. Bim-BH3 concentration to form Mcl-1-Bim-BH3 complex is between 10 uM to 50 uM in these experiments, and a high concentration of Bim may generate unspecific binding to HSMCB. FIG. 15A and FIG. 15B show intracellular flow cytometry measurements in AML biopsies. Patient samples were fixed, permeabilized, stained with HSMCB and anti-Mcl-1 antibody, and measured by flow cytometry. HSMCB/Mcl-1 ratios of blast cells were correlated to patient diagnosis data using TIBCO Spotfire software, white lines represent the median value. (FIG. 15A) HSMCB/Mcl-1 ratio was correlated to FLT3/ITD mutation status. FLT3/ITD positivity (marker for poor prognosis in AML) correlated with low HSMCB/Mcl-1 ratio. (FIG. 15B) HSMCB/Mcl-1 ratio was correlated to disease stage. AML patients with refractory and relapse status had lower HSMCB/Mcl-1 ratio compared to newly diagnosed patients.

### Example 7: A Method for Generating Heterodimer Antibodies

Disclosed herein is a method of isolating, selecting, and purifying a heterodimer antibody (e.g., a MCL-1/BIM heterodimer antibody) from an immunized mouse. The isolation, selection, and purification of a heterodimeric antibody allows for an investigation of the functionality of the heterodimer, such as determining the priming state of a cancer cell, and detecting whether a patient is sensitive to a cancer treatment including with immune modulating drugs. The purified heterodimeric antibodies produced by the methods disclosed herein can be used to detect a heterodimer comprising MCL-1 and BIM proteins in a solid tumor or liquid tumor sample from a patient or a liquid tumor from a patient.

As shown schematically in FIG. 16, a mouse is initially immunized with a covalent heterodimer antigen (e.g., a MCL-1/BIM). A whole cell enzyme-linked immunosorbent assay (ELISA) can be used to test for the presence of the antigen-specific antibody in the immunized mouse serum as well as analyze the antibody titer. Repeated boosters can be performed to increase the antibody titer. An increase in titer is typically observed with each repeated booster. Once a sufficient titer has been achieved (e.g., serum dilutions of up to 1:150,000), the spleen of the mouse is harvested, and the splenic B cells containing the heterodimer are then selected using two affinity-based selection steps: first, the splenic B cells are passed through a magnetic column for negative selection, and then the splenic B cells are passed through a positively charged magnetic column selection. To perform the negative magnetic column-based selection, the splenic B cells are placed onto a negatively charged column that has been coated with both glutathione-derivatized magnetic-beads and a recombinant fusion protein containing one monomer of the heterodimer fused to GST (e.g., a Mcl-1GST). The flow through from the magnetic column for negative selection is then collected, which represents the splenic B cells that did not bind to the monomeric recombinant fusion protein, and thus do not contain the heterodimer. This flow-through containing the B cells from the magnetic column for negative selection is then passed onto a second magnetic column that has been positively coated with the covalent heterodimer antigen (e.g., a Mcl-1/Bim) for positive affinity selection. Cells containing heterodimer-specific antibodies are bound to the magnetic column for positive selection, and then eluted and collected from the positive selection column. The selected cells containing the heterodimer antibodies can then be grown in media with supplements for B cell growth (e.g., IL-4, LPS, and CD40-ligand). Cells can then be isolated and sub-cloned by standard and routine molecular biology methods, and the supernatants can then be screened (e.g., by an ELISA) for antibodies that have superior heterodimer-specific binding and production.

At this stage of the method, the complete sequence of the antibody (e.g., Ig heavy and light chains) that demonstrates the best screening signal (e.g., based on an ELISA) from the supernatants can be identified. For example, the full length of the antibody can be determined using the 5′ or 3′ Race System (i.e., RACE PCR) for rapid amplification of cDNA ends. In these experiments of the method, standard internal primers from the variable region of the mouse heavy and light chain can be used to generate the full length sequence.

Once the optimum heterodimer antibody has been isolated and selected, standard and routine molecular biology methods can be used to clone the isolated heterodimer antibody into an expression vector and expression system (e.g., 293T cells) for purification and large-scale antibody production. The specific binding of the antibody can then be tested in a control assay. For example, a control assay can be an ELISA where the plate has been coated with both the heterodimer antigen (e.g., Mcl-1/Bim, positive) and monomer antigen (e.g., Mcl-1, negative). In some embodiments, the control assay is an immunofluorescence (IF) staining using a cell line that expresses both proteins of the heterodimer (e.g., Mcl-1 and Bim). For example, the IF or IHC staining of a Mcl-1/Bim heterodimer in a cell that expresses both proteins of the Mcl-1/Bim heterodimer can be compared to the IF or IHC staining of a Mcl-1/Bim heterodimer in a different cell that does not express both proteins of the Mcl-1/Bim heterodimer (i.e., the proteins can be knocked down as a control). In some embodiments, the control assay comprises immunohistochemistry (IHC) staining of a cell line that expresses both proteins of the heterodimer (e.g., Mcl-1 and Bim), compared to an IHC staining of a cell line that does not express both proteins of the heterodimer. In some embodiments, the control assay comprises IHC staining on Formalin-Fixed Paraffin-Embedded (FFPE) blocks, which can be derived from a cell line, a control cell line, xenograft tissue, and patient tissue. In some embodiments, the control assay comprises flow cytometry.

In some embodiments, the methods of the present disclosure related to isolating, selecting, and purifying a heterodimer antibody (e.g., a Mcl-1/Bim-BH3 heterodimer antibody) from an immunized mouse can be modified. For example, when the cells containing heterodimer-specific antibodies are eluted and collected from the positive selection column, as described above, the eluted cells containing heterodimer-specific antibodies can be fluorescently labelled (e.g., a fluorescent dye, tag, probe), followed by the culturing of the cells. In some embodiments, the cells are labeled with covalent Mcl-1-GST/Bim BH3-FITC. The labelled cells can then be sorted, for example, by Flow Cytometry and those cells displaying the optimum signal can be gated on the Flow Cytometer and isolated. This step can then be repeated (i.e., culturing of isolated cells from Flow Cytometer, followed by another round of Flow Cytometry), and cells displaying the optimum binding characteristics can be further cloned as described above.

### Example 8: Detection of Shifts in BH3 Priming Signals

In the experiments of this example, a heterodimer specific antibody that recognizes the BCL2:BIM complex (2H3) and detects BH3 mimetic disruption of BCLC2:BIM complexes, allowing for a shift from the BCL2:BIM complex to the MCL-1:BIM complex (FIG. 17). This shift is detected by MCL-1:BIM specific antibody (F05) relative to total MCL-1 in the cell. In this experiment, cells are treated with specific BCL2 targeted BH3 mimetic (AZD4320, Medchemexpress), fixed, permeabilized, and stained with heterodimer-specific antibodies and standard antibodies against monomers. Signals are measured on FLOW Cytometer, show relative signal and the data shown in FIG. 17 is a ratio of complex/monomer signal. FIG. 17 is a graph showing a BCL2:BIM complex specific antibody that tracts with complex disruption by a BH3 mimetic.

FIG. 18 is a graph showing a ratio of measured heterodimer to monomer in Bcl-xL dependent cell lines: Bcl-2:Bim, Mcl-1:Bim, and Bcl-xL:Bim. Bcl-xL:Bim shifts against Bcl-xL and Mcl-1 upon treatment. In this experiment, a shift away from the complex is detected when the sample is treated with BCLXL targeted BH3 mimetic (A1155463). The Mcl-1:Bim complex specific antibody (F05) detects a shift and “priming” to MCL1:BIM complex in A1155463 treated SET2 or UKE-1 cells.

FIG. 19 is a graph showing how sensitivity to selective BH3 mimetics corresponds to PRIMAB readouts. Priming complexes in leukemia cells were measured by intra-cellular immunofluorescent staining with PRIMABs and detected by flow cytometry. BCL-2:BIM specific PRIMAB (HSBXB) detects ABT-199 disruption of BCL2: BIM complex in primed U2932 cells. BCLXL primed U266B1 cells detects BCLXL: BIM shift with HSBXB PRIMAB following treatment with A-1155463 (BCL-XL inhibitor). In (MCL-1 primed H929 cells), MCL1 specific AZD5991 disrupts MCL1:BIM complex detected by HSBXB. These shifts match BH3 mimetic selectivity. This panel of cell lines in the graph legend serves as positive and negative controls for PRIMAB flow assays as described in the legend. These experiments identify and confirm positive control (large priming shift by BH3 mimetic) and negative control (small priming shift), and identify controls for each class of PRIMAB.

FIG. 20A and FIG. 20B are images showing how BH3-mimetics shift priming signals detected by an alternate method aligns with PRIMAB readout. FIG. 20A shows a confirmational measurement of MCL1:BIM Complex. FIG. 20A shows a co-immunoprecipitation experiment of complex from lysates of MCL1 targeting BH3 mimetic (AZD5991) treated or untreated cells, followed by a pull down and ELISA experiment. Resolution with anti-BIM antibody conjugated with Meso Scale Discovery (MSD) SULFOTAG. In FIG. 20B, a signal from treated and untreated whole cells, fixed and stained with PRIMAB F05, and read on flow cytometry is shown. The readout out matched the Co-IP-MSD ELISA readout of BIM in the pull down complex experiment.

Without wishing to be bound by theory, these experiment show that BH3-mimetics shift priming signals detected by an alternate method, and aligns with the PRIMAB readout. The priming complexes can be measured by co-immunoprecipitation followed by western blot or by ELISA. These methods were used to first identify priming complexes and understand their pivotal role in apoptosis. These experiments show that PRIMAB readouts do align with high resolution ELISA detection in “pull down” ELISA.

All of the features disclosed herein may be combined in any combination. Each feature disclosed in this specification may be replaced by an alternative feature serving the same, equivalent, or similar purpose. Thus, unless expressly stated otherwise, each feature disclosed is only an example of a generic series of equivalent or similar features.

From the above description, one skilled in the art can easily ascertain the essential characteristics of the present disclosure, and without departing from the spirit and scope thereof, can make various changes and modifications of the disclosure to adapt it to various usages and conditions. Thus, other embodiments are also within the claims.

What is claimed is:

1. A composition comprising an antibody or antibody format, or fragment thereof, comprising:
(i) a heavy chain variable region comprising heavy chain CDR1, CDR2, and CDR3 sequences, wherein the heavy chain CDR1 sequence is SYAMS (SEQ ID NO: 1), or a variant thereof, the heavy chain CDR2 sequence is TISSGGFATYYPDTVKG (SEQ ID NO: 2), or a variant thereof, and the heavy chain CDR3 sequence is HGGGSYGWFAY (SEQ ID NO: 3), or a variant thereof, and
(ii) a light chain variable region comprising light chain CDR1, CDR2, and CDR3 sequences, wherein the light chain CDR1 sequence is ITSTDIDDDMN (SEQ ID NO: 4), or a variant thereof, the light chain CDR2 sequence is EGNTLRP (SEQ ID NO: 5), or a variant thereof, and the light chain CDR3 sequence is LQSDNMPYT (SEQ ID NO: 6), or a variant thereof.

2. The composition of claim 1, wherein the antibody or antibody format, or fragment thereof, further comprises variable region framework (FW) sequences juxtaposed between the CDRs according to the formula (FW1)-(CDR1)-(FW2)-(CDR2)-(FW3)-(CDR3)-(FW4), wherein the variable region FW sequences in the heavy chain variable region are heavy chain variable region FW sequences, and wherein the variable region FW sequences in the light chain variable region are light chain variable region FW sequences.

3. The composition of claim 2, wherein the variable region FW sequences are human.

4. The composition of any one of claims 1-3, wherein the antibody or antibody format, or fragment thereof, comprises a human heavy chain and light chain constant regions.

5. The composition of any one of claims 1-4, wherein the constant regions are selected from the group consisting of human IgG1, IgG2, IgG3, and IgG4.

6. The composition of any one of claims 1-5, wherein the antibody or antibody format, or fragment thereof, comprises: (i) a heavy chain variable region sequence comprising the amino acid sequence set forth in SEQ ID NO: 7, or the amino acid sequence of SEQ ID NO: 7 having at least about 90% identity thereto; and (ii) a light chain variable region sequence comprising the amino acid sequence of SEQ ID NO: 8, or the amino acid sequence of SEQ ID NO: 8 having at least about 90% identity thereto.

7. The composition of claim 1, wherein the antibody or antibody format, or fragment thereof, comprises an amino acid sequence having at least about 95%, or 97%, or 98% identity with SEQ ID NO: 7 and/or SEQ ID NO. 8.

8. A polynucleotide comprising a nucleic acid sequence encoding the antibody or antibody format, or fragment thereof of any one of claims 1-7.

9. A vector comprising the polynucleotide of claim 8.

10. A host cell comprising the vector of claim 9.

11. A pharmaceutical composition comprising the antibody or antibody format, or fragment thereof, of any one of claims 1-7, and a pharmaceutically acceptable excipient.

12. A composition comprising an antibody or antibody format, or fragment thereof comprising:
(i) a heavy chain variable region comprising heavy chain CDR1, CDR2, and CDR3 sequences, wherein the heavy chain CDR1 sequence is PIAYMS (SEQ ID NO: 9), or a variant thereof, the heavy chain CDR2 sequence is DILPSIGRTIYGEKFED (SEQ ID NO: 10), or a variant thereof, and the heavy chain CDR3 sequence is QDTYYAMDY (SEQ ID NO: 11), or a variant thereof, and
(ii) a light chain variable region comprising light chain CDR1, CDR2, and CDR3 sequences, wherein the light chain CDR1 sequence is SASSSVSYMH (SEQ ID NO: 12), or a variant thereof, the light chain CDR2 sequence is STSNLAS (SEQ ID NO: 13), or a variant thereof, and the light chain CDR3 sequence is QQRSSYPYT (SEQ ID NO: 14), or a variant thereof.

13. The composition of claim 12, wherein the antibody or antibody format, or fragment thereof, further comprises variable region framework (FW) sequences juxtaposed between the CDRs according to the formula (FW1)-(CDR1)-(FW2)-(CDR2)-(FW3)-(CDR3)-(FW4), wherein the variable region FW sequences in the heavy chain variable region are heavy chain variable region FW sequences, and wherein the variable region FW sequences in the light chain variable region are light chain variable region FW sequences.

14. The composition of claim 13, wherein the variable region FW sequences are human.

15. The composition of any one of claims 12-14, wherein the antibody or antibody format, or fragment thereof, comprises a human heavy chain and light chain constant regions.

16. The composition of any one of claims 12-15, wherein the constant regions are selected from the group consisting of human IgG1, IgG2, IgG3, and IgG4.

17. The composition of any one of claims 12-16, wherein the antibody or antibody format, or fragment thereof, comprises: (i) a heavy chain variable region sequence comprising the amino acid sequence set forth in SEQ ID NO: 15, or the amino acid sequence of SEQ ID NO: 15, or an amino acid sequence having at least about 90% identity thereto; and (ii) a light chain variable region sequence comprising the amino acid sequence of SEQ ID NO: 16, or the amino acid sequence of SEQ ID NO: 16 having at least about 90% identity thereto.

18. The composition of claim 12, wherein the antibody or antibody format, or fragment thereof, comprises an amino acid sequence having at least about 95%, or 97%, or 98% identity with SEQ ID NO: 15 and/or SEQ ID NO. 16.

19. A polynucleotide comprising a nucleic acid sequence encoding the antibody or antibody format, or fragment thereof of any one of claims 12-18.

20. A vector comprising the polynucleotide of claim 19.

21. A host cell comprising the vector of claim 20.

22. A pharmaceutical composition comprising the antibody or antibody format, or fragment thereof, of any one of claims 12-18, and a pharmaceutically acceptable excipient.

23. A method for predicting a patient's sensitivity or response to a cancer treatment, comprising:
(a) contacting a sample with an antibody or antibody format, or fragment thereof, from any one of claims 1-18, wherein the antibody recognizes a heterodimer comprising two B-cell lymphoma 2 (BCL-2) proteins selected from MCL-1 and BIM, the sample being a specimen from a solid tumor or liquid tumor of the patient;
(b) detecting a signal that indicates the amount of the heterodimer; and
(c) determining a ratio of the amount of heterodimer present in the sample from step (b) to a reference value,
wherein the reference value comprises the amount of one of the MCL-1 and BIM monomers of the heterodimer in the sample,
the ratio being predictive of the patient's sensitivity to the cancer treatment.

24. A method for predicting a patient's sensitivity or response to a cancer treatment, comprising:
(a) contacting a sample with an antibody or antibody format, or fragment thereof, from any one of claims 1-18, wherein the antibody recognizes a heterodimer comprising two B-cell lymphoma 2 (BCL-2) proteins selected from MCL-1 and BIM, and an antibody or antibody format, or fragment thereof, that recognizes one of the MCL-1 and BIM protein monomers of the heterodimer, the sample being a specimen from a solid tumor or liquid tumor of the patient;
(b) detecting a signal that indicates the amount of the heterodimer and a signal that indicates the amount of the monomer; and
(c) determining a ratio based on the amount heterodimer to the amount of the monomer, the ratio being predictive of the patient's sensitivity to the cancer treatment.

25. The method of claim 23 or 24, further comprising administering a cancer treatment to the patient if the ratio is predictive of sensitivity to the cancer treatment.

26. The method of claim 25, further comprising treating the patient with a reduced dose or less frequent and/or shortened regimen of the cancer treatment if the ratio is predictive of sensitivity to the cancer treatment.

27. The method of claim 25, further comprising treating the patient with an increased dose or more frequent and/or prolonged regimen of the cancer treatment if the ratio is predictive of a lack of sensitivity to the cancer treatment.

28. The method of claim 23 or 24, further comprising withholding cancer treatment from the patient if the ratio is predictive of a lack of sensitivity to the cancer treatment.

29. The method of claim 23 or 24, further comprising treating the patient with a different cancer treatment if the ratio is predictive of a lack of sensitivity to the cancer treatment.

30. The method of any one of claims 23-29, further comprising determining one or more clinical factors of the patient.

31. The method of claim 30, further comprising classifying the patient for likelihood of clinical response to the cancer treatment based on one or more clinical factors of the patient.

32. The method of claim 31, further comprising comparing the prediction of the patient's sensitivity to the cancer treatment with the likelihood of clinical response to the cancer treatment based on one or more clinical factors of the patient.

33. The method of any one of claims 30-32, wherein the clinical factor is one or more of age, cytogenetic status, performance, histological subclass, gender, and disease stage.

34. The method of any one of claims 23-32, further comprising measuring an additional biomarker selected from mutational status, single nucleotide polymorphisms, steady state protein levels, and dynamic protein levels.

35. The method of any one of claims 23-34, wherein the detection of the heterodimer employs an immunohistochemistry (IHC), flow cytometry, or immunofluorescent method.

36. The method of any one of claims 23-35, wherein the method provides a ratio of the MCL-1/BIM heterodimer to one of MCL-1 and BIM monomer.

37. The method of any one of claims 23-36, wherein the cancer treatment comprises a BH3 mimetic.

38. The method of claim 37, wherein the BH3 mimetic is selected from ABT-737 and ABT-263 (navitoclax), Venetoclax (Venclexta, ABT-199), S63845, AMG176, ADZ5991, A-1155463, A1331852, EU5346, or combinations thereof.

39. The method of any one of claims 23-38, wherein the cancer treatment comprises one or more chemotherapy agents.

40. The method of any one of claims 23-39, wherein the cancer treatment is one or more of a SMAC mimetic, proteasome inhibitor, histone deacetylase inhibitor, glucocorticoid, steroid, monoclonal antibody, antibody-drug conjugate, or thalidomide derivative.

41. The method of any one of claims 23-40, wherein the cancer treatment blocks formation of the particular heterodimer detected.

42. The method of any one of claims 23-40, wherein the cancer treatment perturbs or reduces formation of the detected heterodimer.

43. The method of any one of claims 23-36, wherein the cancer treatment comprises a checkpoint inhibitor.

44. The method of claim 43, wherein the checkpoint inhibitor is an agent that targets one of TIM-3, BTLA, PD-1, CTLA-4, B7-H4, GITR, galectin-9, HVEM, PD-L1, PD-L2, B7-H3, CD244, CD160, TIGIT, SIRPα, ICOS, CD172a, and TMIGD2.

45. The method of claim 44, wherein the agent that targets PD-1 is an antibody or antibody format specific for PD-1, optionally selected from nivolumab, pembrolizumab, and pidilizumab.

46. The method of claim 44, wherein the agent that targets PD-L1 is an antibody or antibody format specific for PD-L1, optionally selected from atezolizumab, avelumab, durvalumab, and BMS-936559.

47. The method of claim 44, wherein the agent that targets CTLA-4 is an antibody or antibody format specific for CTLA-4, optionally selected from ipilimumab and tremelimumab.

48. The method of any one of claims 23-47, wherein the sample is selected from a tumor biopsy, tissue biopsy, tumor resection, frozen tumor tissue specimen, lymph node, bone marrow, circulating tumor cells, cultured cells, a formalin-fixed paraffin embedded tumor tissue specimen, bronchoalveolar lavage, skin, hair, urine, and combinations thereof.

49. The method of claim 48, wherein the tumor biopsy is selected from a core biopsy, needle biopsy, surgical biopsy, and an excisional biopsy.

50. The method of any one of claims 23-47, wherein the sample is an infiltrating lymphocyte of the patient.

51. The method of any one of claims 23-47, wherein the solid tumor is selected from lung cancer, breast cancer, prostate cancer, melanoma, pancreatic cancer, kidney cancer, colon cancer, and ovarian cancer.

52. The method of claim 51, wherein the lung cancer is selected from non-small cell lung cancer (NSCLC) and small cell lung cancer (SCLC).

53. The method of claim 51, wherein the breast cancer is triple negative breast cancer.

54. The method of claim 51, wherein the prostate cancer is androgen independent prostate cancer.

55. The method of claim 23, wherein the sensitivity is characterized by a higher likelihood for response to the cancer treatment.

56. The method of any one of claims 23-55, wherein the method does not involve a functional readout of mitochondrial outer membrane permeabilization (MOMP).

57. The method of any one of claims 23-55, wherein the method does not involve a dye-based detection of cell membrane potential.

58. The method of any one of claims 23-57, wherein the antibody or antibody format, or fragment thereof is selected from one or more of a monoclonal antibody, polyclonal antibody, Fab, Fab′, Fab′-SH, F(ab′)2, Fv, single chain Fv, diabody, linear antibody, bispecific antibody, multispecific antibody, chimeric antibody, humanized antibody, human antibody, and a fusion protein comprising the antigen-binding portion of an antibody.

59. The method of any one of claims 23-58, wherein the likelihood of clinical response is defined by the following equation:
$$\% Priming = [ 100 * ( \frac{DMSO AUC - Peptid \text{?} AUC}{DMSO AUC - {CCCP}_{avg} AUC} ) ] Peptid \text{} \text{?} + [ 100 * ( \frac{DMSO AUC - {Peptide}_{2} AUC}{DMSO AUC - {CCCP}_{avg} AUC} ) ] {Peptide}_{2} + … / ( n peptides )$$
$$\text{?} \text{indicates text missing or illegible when filed}$$
wherein:
the AUC (area under a curve) is a sum of fluorescence measurements established by homogenous time-resolved fluorescence (HTRF) or mean signal intensity from fluorescence activated cell sorting (FACS), wherein the signal intensity is a single time point measurement that occurs between about 5 min and about 300 min after the start of priming;
the DMSO (Dimethyl sulfoxide) comprises a baseline negative control for either an area under a curve or a signal intensity;
the CCCP (Carbonyl cyanide m-chlorophenyl hydrazone) is a chemical inhibitor of oxidative phosphorylation and comprises an effector of protein synthesis by serving as uncoupling agent of the proton gradient established during the normal activity of electron carriers in the electron transport chain in the mitochondria, and the CCCP comprises a baseline positive control; and
the Peptide is one or more BH3 domain peptides, wherein (n) is normalized with the average number of replicates of the DMSO and CCCP controls.

60. The method of any one of claims 23-58, wherein the likelihood of clinical response is defined by the following equation:
$$\% Priming = [ 100 * ( \frac{{DMSO}_{avg} AUC - {Peptide}_{n} AUC}{{DMSO}_{avg} AUC - {CCCP}_{avg} AUC} ) ]$$
wherein:
the AUC (area under a curve) is a sum of fluorescence measurements established by homogenous time-resolved fluorescence (HTRF) or mean signal intensity from fluorescence activated cell sorting (FACS), wherein the signal intensity is a single time point measurement that occurs between about 5 min and about 300 min after the start of priming;
the DMSO (Dimethyl sulfoxide) comprises a baseline negative control for either an area under a curve or a signal intensity;
the CCCP (Carbonyl cyanide m-chlorophenyl hydrazone) is a chemical inhibitor of oxidative phosphorylation and comprises an effector of protein synthesis by serving as uncoupling agent of the proton gradient established during the normal activity of electron carriers in the electron transport chain in the mitochondria, and the CCCP comprises a baseline positive control; and
the Peptide is one or more BH3 domain peptides, wherein (n) is normalized with the average number of replicates of the DMSO and CCCP controls.

61. The method of claim 59 or 60, wherein the one or more clinical factors are selected to increase specificity and/or sensitivity of the BH3 profile for association with clinical response.

62. A method for predicting a patient's responsiveness to a checkpoint inhibitor in a sample, comprising measuring the amount of an antibody comprising a MCL-1/BIM heterodimer selected from any one of claims 1-18, wherein the sample comprises an infiltrating lymphocyte population from a solid tumor or liquid tumor.

63. The method of claim 62, wherein the checkpoint inhibitor is an agent that targets one of TIM-3, BTLA, PD-1, CTLA-4, B7-H4, GITR, galectin-9, HVEM, PD-L1, PD-L2, B7-H3, CD244, CD 160, TIGIT, SIRPα, ICOS, CD 172a and TMIGD2.

64. The method of claim 63, wherein the agent that targets PD-1 is an antibody or antibody format specific for PD-1, optionally selected from nivolumab, pembrolizumab, and pidilizumab.

65. The method of claim 63 or 64, wherein the agent that targets PD-L1 is an antibody or antibody format specific for PD-L1, optionally selected from atezolizumab, avelumab, durvalumab, and BMS-936559.

66. The method of claim 63, wherein the agent that targets CTLA-4 is an antibody or antibody format specific for CTLA-4, optionally selected from ipilimumab and tremelimumab.

67. A method of generating a MCL-1 and BIM heterodimer antibody, comprising:
(a) immunizing a subject with a heterodimer induced conformation antigen;
(b) isolating from the subject a splenic B cell producing the IgG recognizing the heterodimer induced antigen;
(c) passing the splenic B cell onto a magnetic column for negative selection, wherein the magnetic column for negative selection is coated with a recombinant fusion protein containing one monomer of the heterodimer;
(d) collecting the flow through of the splenic B cells from the magnetic column for negative selection, and passing the flow through onto a magnetic column for positive selection; wherein the magnetic column for positive selection is coated with the heterodimer antigen;
(e) eluting and collecting the splenic B cells bound to the magnetic column for positive selection;
(f) culturing the collected cells in a B-cell media; and
(g) isolating the heterodimer specific antibody from the cultured cells, thereby generating a heterodimer antibody.

68. The method of claim 67, wherein the subject is a human, a monkey, a mouse, a rat, or a hamster.

69. The composition of any one of claims 1-18, wherein the composition shifts a priming signal away from a BCL2:BIM complex, and towards a MCL-1:BIM complex.

70. The method of any one of claims 24-66, futhering comprising detecting a shift in a priming signal away from a BCL2:BIM complex, and towards a MCL-1:BIM complex.
